(19) United States
(12) Patent Application Publication
(10) Pub. No.: US 2012/0129208 A1
(43) Pub. Date: May 24, 2012

(54) HONEYCOMB SHRINK WELLS FOR STEM CELL CULTURE

(76) Inventors: Michelle Khine, Irvine, CA (US); Diep Nguyen, Merced, CA (US)

(21) Appl. No.: 13/257,016

(22) PCT Filed: Mar. 17, 2010
(86) PCT No.: PCT/US2010/000814

§ 371 Date: Feb. 2, 2012

Related U.S. Application Data
(60) Provisional application No. 61/161,388, filed on Mar. 18, 2009.
(60) Provisional application No. 61/177,871, filed on May 13, 2009.

(51) Int. Cl. C12Q 1/02; C12N 5/07; C12N 5/071; B44C 1/22; B05D 5/00; C23C 16/44; B05D 3/12; C12N 5/00; C12M 1/00

(52) U.S. Cl. 435/29; 435/325; 435/354; 435/366; 435/305.2; 427/277; 427/248.1; 216/83

(57) ABSTRACT

This invention provides a microwell array having a plurality of microwells on a hydrophobic surface wherein the microwells each is substantially proximate to each of its adjacent microwells, as well as methods to prepare arrays. Also provided is a plate that comprises at least one microarray, at least one input channel, at least one output channel, and a channel connecting the input and output channel.

### CROSS-REFERENCE TO RELATED APPLICATIONS

This application claims the benefit under 35 U.S.C. §119(e) of U.S. provisional application Ser. Nos. 61/161,388 and 61/177,871, filed Mar. 18, 2009 and May 13, 2009, respectively, the contents of each of which is hereby incorporated by reference into the present disclosure.

### BACKGROUND OF THE INVENTION

Throughout this disclosure, various technical and patent publications are referenced to more fully describe the state of the art to which this invention pertains. These publications are incorporated by reference, in their entirety, into this application.

Three-dimensional spheroid culture systems (TDSCSs) are well known in the art. Researchers in tumor biology have used TDSCSs to study tumor cell biology, therapy resistance, cell-cell interactions, invasion, drug penetration, modeling, tumor markers, nutrient gradient, and tumor cell metabolism. Other reported uses include the study of numerous cell types such as mammary cells, hepatocytes, bone marrow cells and neural stem and progenitor cells. Paragraph [0003] of US Pat. Pub. 2007/0148767A1. The culturing of embryonic stems cells (ES) is particularly well suited to TDSCS because attachment of the stem cells to the culture surface may cause unwanted differentiation of the cells.

ES cells cultured in vitro will form embryoid bodies (EBs) that contain three germ layers (including mesoderm, endoderm and ectoderm layers). EBs are then further induced to differentiated functional cell types. Prior art methods to produce EBs include the hanging drop technique (Banerjee and Bhonde (2006) Cytotechnology 51(1):1-5), low attachment method (U.S. Pat. No. 6,602,711), liquid bioreactors (Dang et al. (2004) Stem Cells 22(3):275-282), encapsulated liquid suspension culture (Pat. Publ. No. WO 03/004626), semisolid cellulose system (U.S. Patent Publ. No. 2007/0148767A1) and semisolid culture by high viscosity medium and three-dimensional (3-D) culture (Stephen et al. (2002) Biotechnol. Bioeng. 78(4):442-453) and US Pat. Publ. No. 2005/0054100). Dan et al. in “Efficiency of Embryoid Body Formation and Hematopoietic Development from Embryonic Stem Cells in Different Culture System” (2002) Biotechnol. Bioen. 78:442-253, reviews and compares liquid suspension culture, methylcellulose culture, liquid attachment culture and hanging drop to differentiate stem cells to hematopoietic precursor cells.

Successful EB production requires a careful mix of cell and nutrient composition absent adherence of the cells to a surface. The above noted methods provide methods to grow EBs but the methods are not amenable to scale up. Paragraphs [0008] of U.S. Patent Publ. No. 2005/0118711A1. Modifications of these techniques for potential larger scale production of EBs are described in U.S. Patent Publ. Nos. 2005/01187A1; 2006/286666A1; 2008/0026460A1 and 2008/0145925. However, these methods require costly manufacture of devices. In contrast, this method provides a simple, rapid and scalable culture method to load pre-defined cell numbers into microfabricated wells.

### SUMMARY OF THE INVENTION

Embryoid bodies (EB) are aggregates of embryonic stem cells. The most common way of creating these aggregates is the hanging drop method, a laborious approach of pipetting an arbitrary number of cells into well plates. The interactions between the stem cells forced into close proximity of one another promotes the generation of the EBs. Because the media in each of the wells has to be manually exchanged every day, this approach is manually intensive.

Moreover, because environmental parameters including cell-cell, cell-soluble factor interactions, pH, and oxygen availability can be functions of EB size, cell populations obtained from traditional hanging drops can vary dramatically even when cultured under identical conditions. Recent studies have indeed shown that the initial number of cells forming the aggregate can have significant effects on stem cell differentiation. This invention provides an apparatus and simple, rapid, and scalable culture method to load pre-defined numbers of cells into microfabricated wells and maintain them for embryoid body development. Finally, these cells are easily accessible for further analysis and experimentation. This method is amenable to any lab and requires no dedicated equipment.

Thus, in one aspect and referring to FIG. 1, this invention provides a microwell array (1) comprising a plurality of microwells (2, 6, 7, 8) on a hydrophobic surface wherein the microwells each is substantially proximate to each of its adjacent microwells, and the average volume of the microwells is from about 1000 μm³ to about 10 mm³. Also provided and referring to FIG. 3 is a microwell plate (20) comprising one or more of the microwell arrays. The microwell plate (20) may comprise at least one input channel (22) and at least one output channel (24), and a channel (26) connecting the input and output channel. In one aspect, the channel is located such that liquid can be exchanged within the microwells.

Yet further provided is a method for preparing a microwell array comprising a plurality of microwells by: a) applying an image-forming material to a surface of an unstressed or a pre-stressed material in a designed pattern comprising a plurality of filled areas, wherein the filled areas each is substantially proximate to each of its adjacent filled areas and the average area of the filled areas is from about 1000 μm² to about 20 mm²; b) reducing the area of the surface of the thermoplastic material by at least about 60%; and c) preparing the microwell array via lithography on a molded material having a hydrophobic surface. In one aspect, the molded prepared by step c) is washed with solvents. In a further aspect, the resulting surface of the array is pre-treated with a plasma solution to render it hydrophilic. The hydrophilic surface is beneficial to culturing, growing or differentiating cells in the microarrays.

Another aspect for preparing a microwell array is provided herein. This method requires a) etching a designed pattern into a hydrophobic surface of an unstressed or a pre-stressed material, which designed pattern comprises a plurality of filled areas, wherein the filled areas each is substantially proximate to each of its adjacent filled area and the average area of the filled areas is from about 1000 μm² to about 20 mm²; and b) reducing the area of the surface of the unstressed or pre-stressed material by at least about 60%, thereby preparing the microwell array.

Yet further provided is a method for preparing an embryoid body, by placing a solution comprising an isolated embryonic stem cell in a microwell of the microwell array of this invention or the plate of this invention and allowing the cell to settle in the microwell and grow into an embryoid body. In one aspect, the microwell has been pre-treated with a solution such as a plasma solution to render the well hydrophilic. In a further aspect, the embryoid body is removed from the microwell after culture. In a further aspect the cell is cultured and differentiated into a cardiomyocyte.

This invention further provides a method for assaying a potential agent for the ability to affect growth and/or differentiation of an isolated stem cell, by placing a solution comprising an isolated stem cell and an agent in a microwell of the microwell plate as described herein and allowing the cell to settle on the plate and grow and/or differentiate; and assaying for the agent's ability to affect growth and/or differentiation of the cell.

### BRIEF DESCRIPTION OF THE FIGURES

FIG. 1 shows topical views of various embodiments of the honeycomb microwells.

FIG. 2 shows a side view of the honeycomb microarray.

FIG. 3 shows a plate (20) containing microwells having input (22), output (24) and a channel (26) connecting them.

FIG. 4 also shows a plate having the microwell arrays and the growing of embryoid bodies in the wells of the microarray.

FIG. 5 is a close-up of the cells in the wells of the microarray.

FIG. 6 illustrates a honeycomb microwell fabrication. FIG. 6A shows microwell patterns of tunable sizes are printed on pre-stressed PS sheets (1) and are then heated to 155° C. (2) for approximately 5 minutes to form high-aspect micromolds. PDMS is then molded (3) and removed (4) and cells are pipetted in (5). FIG. 6B shows PDMS molded onto the PS masters forming microwells. The bottom-side of the microwells are then bonded to glass slides (to prevent floating) and inserted into standard culture plates.

FIG. 6C shows, in addition, the fabricated wells have tunable rounded bottoms which facilitate aggregation of single-cells; cross section of Small, Medium and Large wells.

FIG. 7 illustrates a honeycomb microwell characterization. FIG. 7A shows characteristic change of microwell diameters and depth with repeated prints for wells with drafting diameters of 250, 500, 750, and 890 μm. FIG. 7B shows calibration of loading concentrations 1.39×10⁴ cells/mL, 4.17×10⁴ cells/mL, 1.25×10⁵ cells/mL, and 3.75×10⁵ cells/mL corresponding to Small, Medium and Large wells. Using the loading concentration of 1.25×10⁵ cells/mL, aggregate sizes were characterized on day 2 for Small, Medium and Large wells. Standard error of mean was calculated for N=25 per concentration and aggregate size, and N=10 per depth and diameter measurement within the family curves. FIG. 7C shows uniform aggregates from a higher seeding concentration of 3.75×10⁵ cells/mL in large wells results in uniform EBs similar in size to the hanging drop method.

FIG. 8 shows that EB differentiation occurs normally on chip as indicated by Oct-3/4 expression. Each separate sample of EBs derived from Small (Red), Medium (Green), and Large (Blue) wells corresponding to initial mESC aggregates of 130, 220, and 445 cells±15, 30, and 56 cells respectively. FIG. 8A shows day 0 mESC Oct-3/4 expression. FIG. 8B shows Oct-3/4 expression of EBs transferred on day 2. FIG. 8C shows Oct-3/4 expression of day 4 EBs, 2 days post transfer. FIG. 8D shows Oct-3/4 expression of day 6 EBs, 4 days post transfer. As control, all expressions are relative to unstained permeabilized cells.

FIG. 9 shows EBs derived from microwells and transferred on day 2 to suspension culture plates. FIG. 9A shows day 4 EBs in suspension of the 3 different sized wells show morphologically properly developing EBs. FIG. 9B shows day 6 EBs in suspension develops cystic-like morphology.

FIG. 10 illustrates EB Markers by FACS analysis. FIG. 10A shows time course expression of GATA4, Nestin and CD-31 from EBs derived from Small (Red), Medium (Green) and Large (Blue) wells. GATA4 expression is upregulated by day 4 with medium-sized EB populations derived from initial aggregates of approximately 220 initial cells showing highest expression. GATA4 expression is upregulated in all populations by day 6. Nestin expression is upregulated by day 4, indicative of ectodermal layer. Small EBs populations derived from initial aggregates of approximately 130 cells show preferentially high expression of Nestin by day 4 relative to GATA4 and CD-31. By day 6, Nestin is uniformly upregulated in all three populations. CD-31 is detected in the starting mESC population as indicative of undifferentiated cells and is downregulated by day 4 in the small and medium populations more so than in the large EBs. By day 6 CD-31 is uniformly downregulated across all three populations. FIG. 10B shows the time course percent expression of GATA4, Nestin, and CD-31 for Small, Medium and Large microwells. All expression levels are relative to the control, unstained permeabilized cells.

FIG. 11 illustrates beating EBs derived from microwells. FIG. 11A shows EBs derived from large wells seeded at density of 3.75×10⁵ cells/mL develops into beating cardiomyocyte by day 14 as detected by GFP-labeled myosin heavy chain reporter gene. EBs were transferred from suspension culture onto gelatin coated culture plates at day 2. FIG. 11B shows beating EBs derived from the traditional hanging drop method shows similar GFP expression.

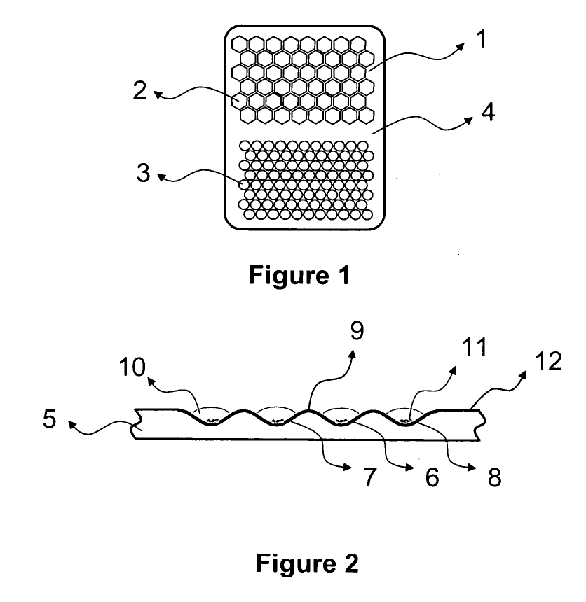

### DETAILED DESCRIPTION OF THE INVENTION

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis, Molecular Cloning: A Laboratory Manual, 2^{nd} edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Harlow and Lane, eds. (1999) Using Antibodies, A Laboratory Manual; and Animal Cell Culture (R. I. Freshney, ed. (1987)).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used herein, certain terms may have the following defined meanings.

As used in the specification and claims, the singular form “a,” “an” and “the” include plural references unless the context clearly dictates otherwise. For example, the term “a microwell” includes a plurality of microwells.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps for preparing the microarray and device. Embodiments defined by each of these transition terms are within the scope of this invention.

A “thermoplastic material” is intended to mean a plastic material which shrinks upon heating. In one aspect, the thermoplastic materials are those which shrink uniformly without distortion. A “Shrinky-Dink” is a commercial thermoplastic which is used a children's toy. The shrinking can be either bi-axially (isotropic) or uni-axial (anisotropic) stressed. Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon. As used herein, the terms “thermoplastic base” and “thermoplastic cover” refer to thermoplastic material having been subjected to both the etching process as well as heating process. The “thermoplastic base” would be located at the bottom or within the device, and the “thermoplastic cover” is the last layer of one or more layers of thermoplastic base.

A “well” is intended to mean a depression which is disposed within one or more levels of the microwell structure. The term “microwell” is generally defined as a substrate or material having a fluid depression with at least one internal cross-sectional dimension which can be used in any number of biochemical or biological processes involving very small amounts of fluid. Such processes include, but are not limited to, containing and/or propagating cell compositions such as stem cells as described herein.

A “channel” is intended to mean a flow path which is disposed between the microwells. The term “microfluidic” is generally defined as a substrate or material having a passage through which a fluid, solid or gas can pass with at least one internal cross-sectional dimension that is less than about 500 micrometers and typically between about 0.1 micrometers and about 500 micrometers which can be used in any number of chemical processes involving very small amounts of material fluid. Such processes include, but are not limited to, electrophoresis (e.g., capillary electrophoresis or CE), chromatography (e.g., liquid chromatography), screening and diagnostics (using, e.g., hybridization or other binding means), and chemical and biochemical synthesis (e.g., DNA amplification as may be conducted using the polymerase chain reaction, or “PCR”) and analysis (e.g., through enzymatic digestion).

In addition to the above uses, the microfluidic channels disclosed herein can be patterned for “microfluidic mixing.” As used herein, the term “microfluidic mixing” is intended to mean the use of a receptive material having at least two inlet channels, wherein the inlet channels meet or intersect at an overlap region that may be in fluid communication with an outlet channel, such that fluids, such as solutions or other material, introduced from the inlet channels are mixed and may proceed into an outlet channel.

A “solution” is intended to refer to a substantially homogeneous mixture of a solute, such as a solid, liquid, or gaseous substance, with a solvent, which is typically a liquid. The solution can be either aqueous or non-aqueous. Examples of suitable solutes in solutions include fluorescent dyes, biological compounds, such as proteins, DNA and plasma, and soluble chemical compounds. Examples of suitable solids include beads, such as polystyrene beads, and powders, such as a metal powder. A “suspension” is intended to refer to a substantially heterogeneous fluid containing a solid, wherein the solid is dispersed throughout the liquid, but does not substantially dissolve. The solid particles in a suspension will typically settle as the particle size is large, compared to a colloid, where the particle size is small such that the suspension does not settle. Examples of suitable suspensions include biological suspensions such as whole blood, cell compositions, or other cell containing mixtures. It is contemplated that any solution, solid or suspension can be mixed using the mixers disclosed herein, provided that the solid has a particle size sufficiently small to move throughout the channels in the mixer.

An “effective amount” is an amount sufficient to effect beneficial or desired results. An effective amount can be administered in one or more administrations, applications or dosages.

A “control” is an alternative subject or sample used in an experiment for comparison purpose. A control can be “positive” or “negative.”

A “pharmaceutical composition” is intended to include the combination of an active agent with a carrier, inert or active such as a biocompatible scaffold, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)). The term includes carriers that facilitate controlled release of the active agent as well as immediate release.

In general, the image-forming material is one which is compressed upon heating, bonds to the plastic and is durable (can be used as a mold for multiple iterations). For example, “image-forming material” is, in one aspect, intended to mean a composition, typically a liquid, containing various pigments and/or dyes used for coloring a surface to produce an image or text such as ink and printer toner. In addition to an ink, the image forming material can be a metal, such as gold, titanium, silver, a protein, a colloid, a dielectric substance, a paste or any other suitable metal or combination thereof. Examples of suitable proteins include biotin, fibronectin and collagen. Examples of suitable colloids include pigmented ink, paints and other systems involving small particles of one substance suspended in another. Examples of suitable dielectric substances include metal oxides, such as aluminum oxide, titanium dioxide and silicon dioxide. Examples of suitable pastes include conductive pastes such as silver pastes.

The image forming material can be applied to the thermoplastic material by a variety of methods known to one skilled in the art, such as printing, sputtering and evaporating. The term “evaporating” is intended to mean thermal evaporation, which is a physical vapor deposition method to deposit a thin film of metal on the surface of a substrate. By heating a metal in a vacuum chamber to a hot enough temperature, the vapor pressure of the metal becomes significant and the metal evaporated. It recondenses on the target substrate. As used herein, the term “sputtering” is intended to mean a physical vapor deposition method where atoms in the target material are ejected into the gas phase by high-energy ions and then land on the substrate to create the thin film of metal. Such methods are well known in the art (Bowden et al. (1998) Nature (London) 393:146-149; Bowden et al. (1999) Appl. Phys. Lett. 752557-2559; Yoo et al. (2002) Adv. Mater. 14:1383-1387; Huck et al. (2000) Langmuir 16:3497-3501; Watanabe et al. (2004) J. Polym. Sci. Part B: Polym. Phys. 42:2460-2466; Volynskii et al. (2000) J. Mater. Sci. 35:547-554; Stafford et al. (2004) Nature Mater. 3:545-550; Watanabe et al. (2005) J. Polym. Sci. Part B: Polym. Phys. 43:1532-1537; Lacour, et al. (2003) Appl. Phys. Lett. 82:2404-2406.)

In addition, the image forming material can be applied to the thermoplastic material using “pattern transfer”. The term “pattern transfer” refers to the process of contacting an image-forming device, such as a mold or stamp, containing the desired pattern with an image-forming material to the thermoplastic material. After releasing the mold, the pattern is transferred to the thermoplastic material. In general, high aspect ratio pattern and sub-nanometer patterns have been demonstrated. Such methods are well known in the art (Sakurai, et al., U.S. Pat. No. 7,412,926; Peterman, et al., U.S. Pat. No. 7,382,449; Nakamura, et al., U.S. Pat. No. 7,362,524; Tamada, U.S. Pat. No. 6,869,735).

Another method for applying the image forming material includes, for example “micro-contact printing.” The term “micro-contact printing” refers to the use of the relief patterns on a PDMS stamp to form patterns of self-assembled monolayers (SAMs) of an image-forming material on the surface of a thermoplastic material through conformal contact. Micro-contact printing differs from other printing methods, like inkjet printing or 3D printing, in the use of self-assembly (especially, the use of SAMs) to form micro patterns and microstructures of various image-forming materials. Such methods are well known in the art (Cracauer, et al., U.S. Pat. No. 6,981,445; Fujihira, et al., U.S. Pat. No. 6,868,786; Hall, et al., U.S. Pat. No. 6,792,856; Maracas, et al., U.S. Pat. No. 5,937,758).

“Soft-lithography” is intended to refer to a technique commonly known in the art. Soft-lithography uses a patterning device, such as a stamp, a mold or mask, having a transfer surface comprising a well defined pattern in conjunction with a receptive or conformable material to receive the transferred pattern. Microsized and nanosized structures are formed by material processing involving conformal contact on a molecular scale between the substrate and the transfer surface of the patterning device.

The term “receptive material” is intended to refer to a material which is capable of receiving a transferred pattern. In certain embodiments, the receptive material is a conformable material such as those typically used in soft lithography comprise of elastomeric materials, such as polydimethylsiloxane (PDMS). The thermoplastic receptive material, or thermoplastic material, is also a receptive material as it can be etched, for example.

“Imprint lithography” is intended to refer to a technique commonly known in the art. “Imprint lithography” typically refers to a three-dimensional patterning method which utilizes a patterning device, such as a stamp, a mold or mask.

A “mold” is intended to mean an imprint lithographic mold.

A “patterning device” is intended to be broadly interpreted as referring to a device that can be used to convey a patterned cross-section, corresponding to a pattern that is to be created in a target portion of the substrate.

A “pattern” is intended to mean a mark or design.

“Bonded” is intended to mean a fabrication process that joins materials, usually metals or thermoplastics, by causing coalescence. This is often done by melting the materials to form a pool of molten material that cools to become a strong joint, with pressure sometimes used in conjunction with heat, or by itself, to produce the bond.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

As used herein, “stem cell” defines a cell with the ability to divide for indefinite periods in culture and give rise to specialized cells. At this time and for convenience, stem cells are categorized as somatic (adult) or embryonic. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. An embryonic stem cell is one that has been cultured under in vitro conditions that allow proliferation without differentiation for months to years. Non-limiting examples of embryonic stem cells are the HES2 (also known as ES02) cell line available from ESI, Singapore and the H1 (also know as WA01) cell line available from WiCells, Madison, Wis. Methods to prepare hESC are known in the art and described, for example in Xue et al. (2005) Circulation 111:11-20, Thomson et al. (1998) Science 282:1145-1147, Moore et al. (2005) Reproductive Toxicology 20:377-391, and Wang et al. (2005) Stem Cells 23:1526-1534. Available sources of these cells include, for example, from the NIH Human Embryonic Stem Cell Registry. Pluripotent embryonic stem cells can be distinguished from other types of cells by the use of marker including, but not limited to, Oct-4, alkaline phosphatase, CD30, TDGF-1, GCTM-2, Genesis, Germ cell nuclear factor, SSEA1, SSEA3, and SSEA4. A clone is a line of cells that is genetically identical to the originating cell; in this case, a stem cell.

In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes.

Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi K. et al. (2007) Cell advance online publication 20 Nov. 2007; Takahashi K. & Yamanaka S. (2006) Cell 126: 663-76; Okita K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A clone is a line of cells that is genetically identical to the originating cell; in this case, a stem cell. “Clonal proliferation” refers to the growth of a population of cells by the continuous division of single cells into two identical daughter cells and/or population of identical cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of an embryoid body.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

As used herein, the “lineage” of a cell defines the heredity of the cell, i.e. its predecessors and progeny. The lineage of a cell places the cell within a hereditary scheme of development and differentiation.

A derivative of a cell or population of cells is a daughter cell of the isolated cell or population of cells. Derivatives include the expanded clonal cells or differentiated cells cultured and propagated from the isolated stem cell or population of stem cells. Derivatives also include already derived stem cells or population of stem cells, such as, embryoid bodies from an embryonic stem cell.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

Examples of cells that differentiate into endodermal lineage include, but are not limited to pleurogenic cells, and hepatogenic cells, cell that give rise to the lining of the intestine, and cells that give rise to pancreogenic and splanchogenic cells.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi K. et al. (2007) Cell advance online publication 20 Nov. 2007; Takahashi K. & Yamanaka S. (2006) Cell 126:663-76; Okita K. et al. (2007) Nature 448:260-262; Yu, J. et al. (2007) Science advance online publication 20 Nov. 2007; and Nakagawa, M. et al. (2007) Nat. Biotechnol. Advance online publication 30 Nov. 2007.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

“Embryoid bodies or EBs” are three-dimensional (3-D) aggregates of embryonic stem cells formed during culture that facilitate subsequent differentiation. When grown in suspension culture, ES cells form small aggregates of cells surrounded by an outer layer of visceral endoderm. Upon growth and differentiation, EBs develop into cystic embryoid bodies with fluid-filled cavities and an inner layer of ectoderm-like cells.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker, e.g. myosin or actin or the expression of a gene or protein, e.g. a calcium handling protein, a t-tubule protein or alternatively, a calcium pump protein. In another aspects, the substantially homogenous population have a decreased (e.g., less than about 95%, or alternatively less than about 90%, or alternatively less than about 80%, or alternatively less than about 75%, or alternatively less than about 70%, or alternatively less than about 65%, or alternatively less than about 60%, or alternatively less than about 55%, or alternatively less than about 50%) of the normal level of expression than the wild-type counterpart cell or tissue.

A “composition” is intended to mean a combination of active agent, cell or population of cells and another compound or composition, inert (for example, a detectable agent or label) or active, such as a biocompatible scaffold.

A “subject,” “individual” or “patient” is used interchangeably herein, and refers to a vertebrate, preferably a mammal, more preferably a human. Mammals include, but are not limited to, murines, rats, simians, bovines, canines, humans, farm animals, sport animals and pets. The cells useful in this invention can be from any appropriate subject, such as a mouse (murine) or a human patient.

### Honeycomb Microwell Arrays

This invention provides a microwell array (1) comprising, or alternatively consisting of, or yet further consisting of a plurality of microwells (2, 6, 7, 8) on a hydrophobic surface wherein the each of the microwell is substantially proximate to each of its adjacent microwells, and the average volume of the microwells is from about 1000 mm³ to about 10 mm³, or alternatively from about 900 μm³ to about 9 mm³, or alternatively from about 800 μm³ to about 8 mm³, or alternatively from about 700 μm³ to about 7 mm³, or alternatively from about 600 μm³ to about 6 mm³, or alternatively from about 500 μm³ to about 5 mm³ or alternatively from about 400 mm³ to about 4 mm³, or alternatively from about 300 μm³ to about 3 mm³, or alternatively from about 200 μm³ to about 20 mm³ or alternatively from about 100 μm³ to about 1 mm³ or alternatively less than about from about 1000 μm³, or alternatively less than about 900 μm³, or alternatively less than about 800 μm³, or alternatively less than about 700 μm³, or alternatively less than about 600 μm³, or alternatively less than about 500 μm³, or alternatively less than about 400 μm³, or alternatively less than 300 μm³, or alternatively less than about 200 μm³, or alternatively less than about 100 μm³, or alternatively less than about 100 mm³, or alternatively less than about 50 mm³. Stated another way, the microwells will have a well diameter of about 0.0175″, or about 0.0148″, or about 0.0099″, or about 0.0049″ respectively with the spacing of the centers of each well at about 0.0179″, or about 0.0450″, or about 0.0147″, or about 0.0117″, respectively.

In one aspect, in the microwell array, is made of a receptive, conformable material such as those typically used in soft lithography which comprise, or alternatively consists essentially of, or yet further consists of one or more elastomeric materials, such as polydimethylsiloxane, gelatin, agarose, polyethylene glycol, cellulose nitrate, polyacrylamide or chitosan. Alternatively, the microwell array is made from one or more thermoplastic materials from the group consisting of acrylonitrile butadiene styrene, acrylic, celluloid, cellulose acetate, ethylene-vinyl acetate, ethylene vinyl alcohol, fluoroplastics, ionomers kydex, a trademarked acrylic/PVC alloy, liquid crystal polymer, polyacetal, polyacrylates, polyacrylonitrile, polyamide, polyamide-imide, polyaryletherketone, polybutadiene, polybutylene, polybutylene terephthalate, polyethylene terephthalate, Polycyclohexylene Dimethylene Terephthalate, polycarbonate, polyhydroxyalkanoates, polyketone, polyester polyethylene, polyetheretherketone, polyetherimide, polyethersulfone, polysulfone polyethylenechlorinates, polyimide, polylactic acid, polymethylpentene, polyphenylene oxide, polyphenylene sulfide, polyphthalamide, polypropylene, polystyrene, polysulfone, polyvinyl chloride, polyvinylidene chloride, and spectralon. In one particular embodiment, the receptive material is polystyrene.

After creating the microwell array, the array may be washed in a series of solvents as described in more detail in the experimental section below.

In a further aspect, the surface of the hydrophobic surface of the microarray lacks surface tension which is accomplished by the close proximity of the wells to each other and the composition of the underlying material of the array.

The microwells of the array are in close proximity to each other such that each of the microwells has a distance to an adjacent microwell, wherein the distance is substantially the same as or slightly greater than the sum of the radii of both of the microwells.

The microwells of the array have substantially the same or different volumes. For the purpose of illustration only, the volume of each microwell can be from about 1×10⁶ μm³ to about 0.2 μm³, or alternatively from about 1×10⁵ μm³ to about 0.2 mm³, or alternatively from about 1×10⁴ μm³ to about 0.2 μm³, or alternatively from about 1×10³ mm³ to about 0.2 μm³, or alternatively from about 1×10² μm³ to about 0.2 mm³, or alternatively from about 10 μm³ to about 0.2 mm³, or alternatively from about 1 μm³ to about 0.2 μm³, or alternatively from about 0.5 μm³ to about 0.2 μm³, or alternatively less than about 1×10⁶ mm³, or alternatively less than about 1×10⁵ μm³, or alternatively less than about 1×10⁴ μm³, or alternatively less than about 1×10³ μm³, or alternatively less than about 1×10² μm³, or alternatively from about 10 μm³, or alternatively less than about 1 μm³.

In another aspect, each of the microwells has a diameter of from about 10 micrometers to about 2 millimeters, or alternatively from about 9 micrometers to about 2 millimeters, or alternatively from about 8 micrometers to about 2 millimeters, or alternatively from about 7 micrometers to about 2 millimeters, or alternatively from about 6 micrometers to about 2 millimeters, or alternatively from about 5 micrometers to about 2 millimeters, or alternatively from about 4 micrometers to about 2 millimeters, or alternatively less than about 9 micrometers, or alternatively less than about 8 micrometers, or alternatively less than about 7 micrometers, or alternatively less than about 6 micrometers, or alternatively less than about 5 micrometers, or alternatively less than about 4 micrometers, or alternatively less than about 3 micrometers.

In another aspect, the each of the microwells has a diameter of from about 100 micrometers to about 500 micrometers, or alternatively from about 200 micrometers to about 500 micrometers, or alternatively from about 300 micrometers to about 500 micrometers, or alternatively from about 100 micrometers to about 400 micrometers, or alternatively from about 100 micrometers to about 300 micrometers. In a yet further aspect, each of the microwells has the same or different diameter and is selected from the group consisting of about 100 micrometers, about 200 micrometers, about 300 micrometers, about 400 micrometers, and about 500 micrometers.

With each of the above diameters for the microwells, the microwells can be the same or different depths. The microwells each may have a depth of from about 10 micrometers to about 2 millimeters, 1 or alternatively from about 9 micrometers to about 2 millimeters, or alternatively from about 8 micrometers to about 2 millimeters, or alternatively from about 7 micrometers to about 2 millimeters, or alternatively from about 6 micrometers to about 2 millimeters, or alternatively from about 5 micrometers to about 2 millimeters, or alternatively from about 4 micrometers to about 2 millimeters, or alternatively less than about 9 micrometers, or alternatively less than about 8 micrometers, or alternatively less than about 7 micrometers, or alternatively less than about 6 micrometers, or alternatively less than about 5 micrometers, or alternatively less than about 4 micrometers, or alternatively less than about 3 micrometers, or alternatively of from about 100 micrometers to about 500 micrometers, or alternatively from about 200 micrometers to about 500 micrometers, or alternatively from about 300 micrometers to about 500 micrometers, or alternatively from about 100 micrometers to about 400 micrometers, or alternatively from about 100 micrometers to about 300 micrometers. In a yet further aspect, each of the microwells has the same or different diameter and is selected from the group consisting of about 100 micrometers, about 200 micrometers, about 300 micrometers, about 400 micrometers, and about 500 micrometers.

In each of the above embodiments, the microwells can have substantially identical diameters or identical diameters, or alternatively substantially identical depths or alternatively identical depths.

The shape of the microwells can vary. The microwells can each have a contour selected from the group consisting of substantially hexangle, substantially heptangle, substantially octangle, and substantially round and the shapes can be substantially identical contours or not all of the microwells can have identical contours. In one specific embodiment, the microwells are organized in a honeycomb pattern.

This invention also provides the microwell array wherein the array is made of a receptive material, which may be in one aspect, an unstressed or pre-stressed or heat-shrunk thermoplastic material. Alternatively, the receptive material is selected from the group consisting of acrylonitrile butadiene styrene, acrylic, celluloid, cellulose acetate, ethylene-vinyl acetate, ethylene vinyl alcohol, fluoroplastics, ionomers kydex, a trademarked acrylic/PVC alloy, liquid crystal polymer, polyacetal, polyacrylates, polyacrylonitrile, polyamide, polyamide-imide, polyaryletherketone, polybutadiene, polybutylene, polybutylene terephthalate, polyethylene terephthalate, Polycyclohexylene Dimethylene Terephthalate, polycarbonate, polyhydroxyalkanoates, polyketone, polyester polyethylene, polyetheretherketone, polyetherimide, polyethersulfone, polysulfone polyethylenechlorinates, polyimide, polylactic acid, polymethylpentene, polyphenylene oxide, polyphenylene sulfide, polyphthalamide, polypropylene, polystyrene, polysulfone, polyvinyl chloride, polyvinylidene chloride, and spectralon. In one particular embodiment, the receptive material is polystyrene. The pre-stressed receptive material may be uni-axially biased or bi-axialy biased.

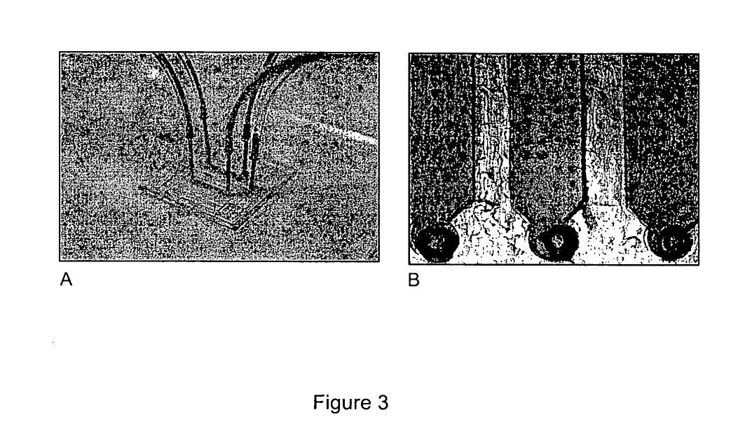

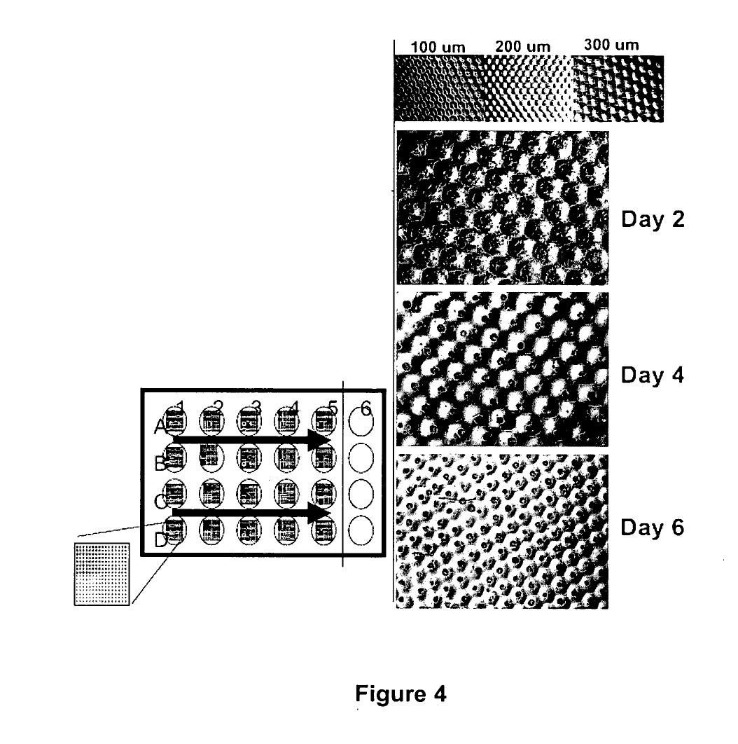

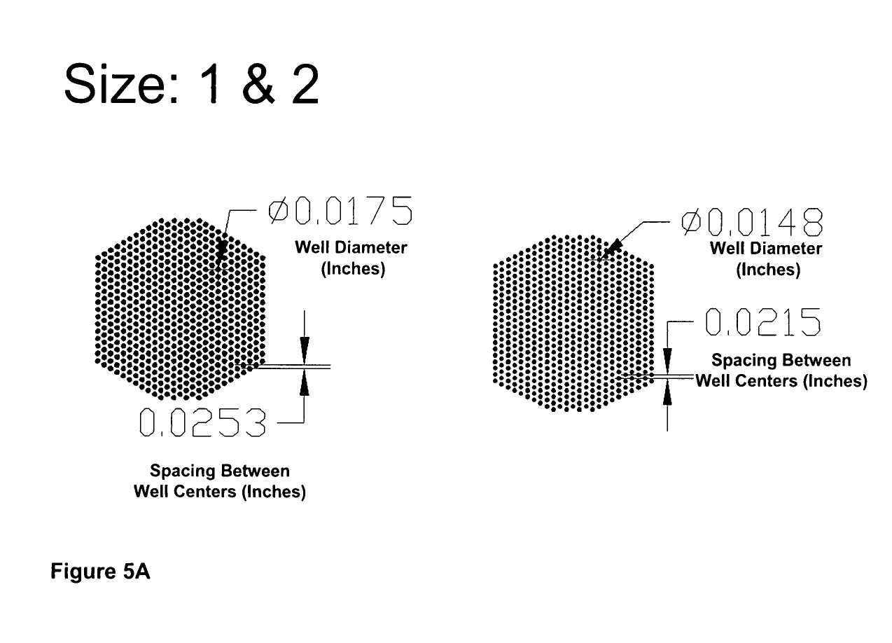

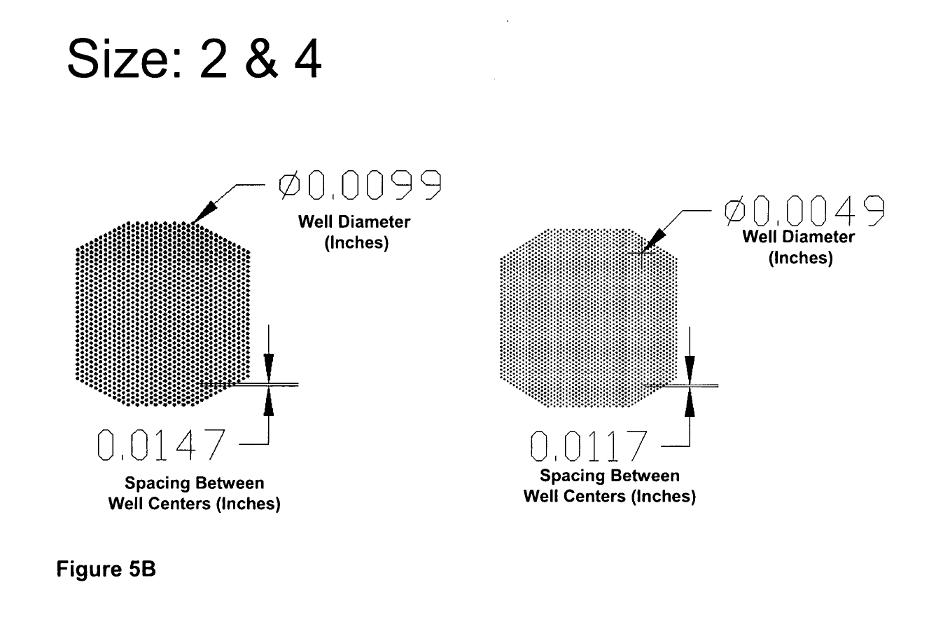

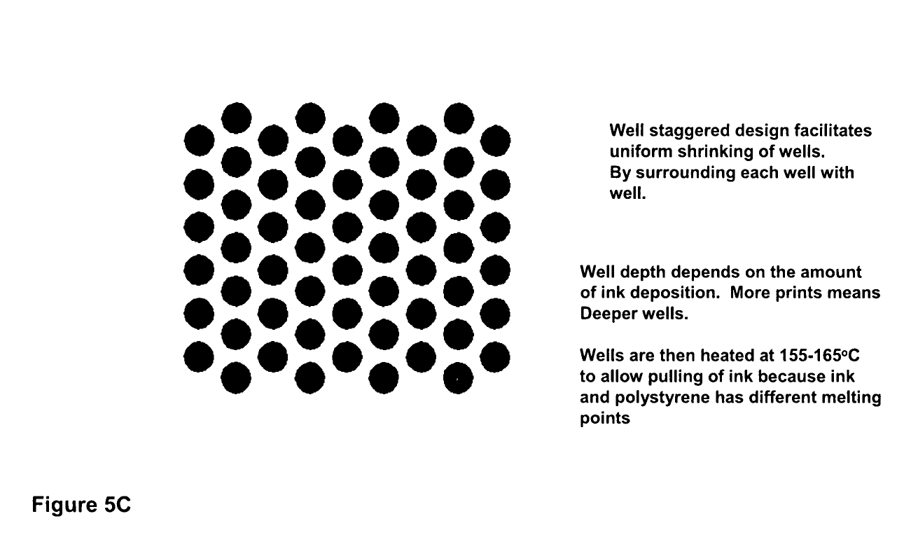

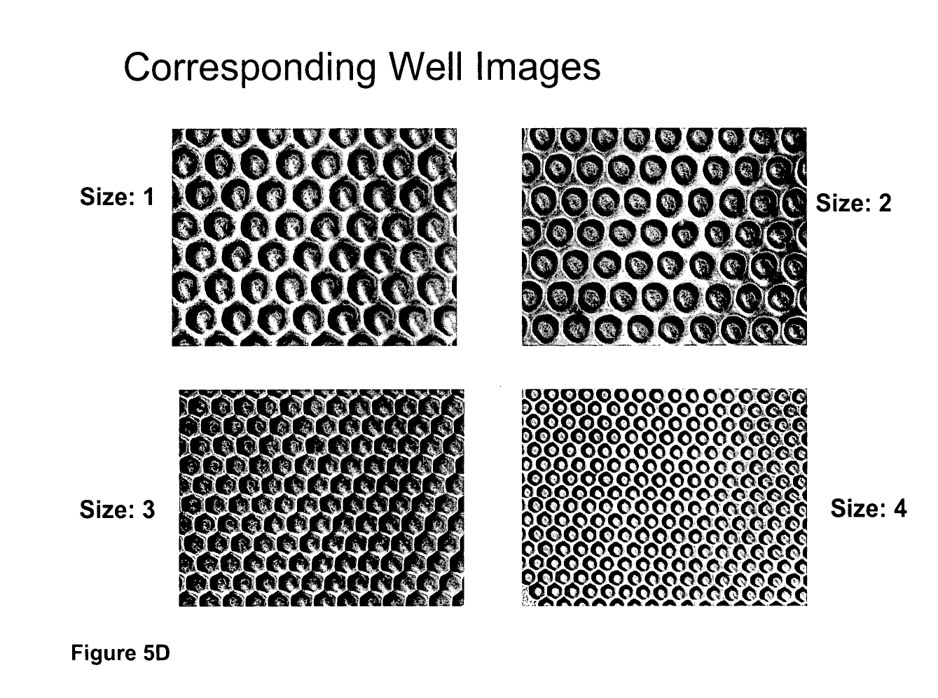

Also provided by this invention is a microwell plate (20) having one or more of the microwell arrays as described above. The plate is shown schematically in FIGS. 3 and 4.

### Methods for Preparing and Using Microwells

Methods have been developed as lower-cost alternatives to photolithography, the ‘gold standard’ for microfabrication and microfluidic device creation. Duffy et al. first introduced ‘rapid prototyping of masters’ whereby they used printed transparencies to replace the expensive chrome masks traditionally utilized in photolithography (Duffy D., et al. (1998) Anal Chem. 70:4974-4984). The authors demonstrated the advantages of using rapid prototyping for masks over conventional photolithography and micromachining. Despite its convenience, the method still requires the use of expensive photoresist, high-resolution printing, and contact lithography. Tan et al. obviated this issue by direct printing; they photocopied designs onto transparencies to fabricate microfluidic channel molds that ranged in height from 8-14 micrometer, depending on the darkness setting of the photocopy machine (Tan A., et al. (2001) Lab Chip 1:7-9). Liu et al. developed a one-step direct-printing technique for the design and fabrication of passive micromixers in microfluidic devices, with a maximum channel height of 11 micrometer (Liu A., et al. (2005) Lab Chip 5:974-978). Such shallow channels are adequate for many microfluidic applications but not amenable for use with large mammalian cells (>10 micrometer in diameter) as well as other applications, such as flowing chemotactic gradients across adherent cells in a channel with minimal shearing (Lin F., et al. (2004) Biochem. And Biophys. Res. Commun. 319:576-581).

While Lago et al. introduced a way to circumvent the height limitation of single-layer ink by printing up to four times using a thermal toner transfer method onto a glass substrate, the maximum height obtained with this approach was 25 micrometer (Lago C. L., et al. (2004) Electrophoresis 25:3825-3831). Vullev et al. demonstrated a non-lithographic fabrication approach of microfluidic devices by printing positive-relief masters with a laser-jet printer for detecting bacterial spores; the height of the channels, which is likewise dependent on the height of the ink, is limited to between 5-9 micrometer (Vullev V., et al. (2006) J. Am. Chem. Soc. 128:16062-16072). To achieve deep channels, McDonald et al. introduced the use of solid object printing (SOP) to make PDMS molds in thermoplastics (McDonald J. C., et al. (2002) Anal. Chem. 74:1537-1545). However, despite their versatility, solid object printers are considerably costly ($50,000.)

Furthermore, the majority of these methods (as well as conventional photolithography) produce rectangular cross section channels. Pneumatic valves, first introduced by Quake et al., important for many microfluidic applications, require microfluidic channels to be rounded such that they can be completely sealed upon valve closure (Unger M. A., et al. (2000) Science 288(5463):113-116). Achieving rounded microfluidic channels using typical photolithographic techniques, however, is complicated and requires an extra re-flow step of the photoresist at high temperatures. Most recently, Chao et al. demonstrated an elegant rapid prototyping approach, coined microscale plasma templating (μPLAT), using water molds. This technique enables the creation of rounded channels that are difficult to make with photolithography, but still requires micromachined masks and plasma activation (Chao S. H., et al. (2007) Lab Chip Technical Note 7:641-643).

In one aspect, this invention is a method to prepare a plurality of wells by applying a first image-forming material such as ink, to a second unstressed or pre-stressed thermoplastic material in designed well type pattern and then heating the first and second material under conditions that reduce the length and width of the second thermoplastic material by at least 20% and increase the thickness by at least 120%, or alternatively at least 130%, or alternatively, at least 140%, or alternatively at least 150%, of the area of the second thermoplastic material to which the first lithographic material is applied, thereby producing a mold. Thereafter, preparing the plurality of wells on a third molding material is prepared via a procedure such as molding using the thermoplastic material.

Suitable first image-forming material includes without limitation an ink, metal, protein, biodegradable material, fluorescent dye, battery material, polymer, or conductive polymer. The first material can be applied to the thermoplastic material by sputtering, evaporating, printing, depositing, or stamping. Applicants have successfully used commercially available inkjet and laser jet printers to transfer the first material to the second. The transfer of the first material may be performed in one or more steps prior to heating the second material to reduce its size.

The thickness of the image-forming material, such as ink or toner, onto the heat sensitive thermoplastic receptive material dictates the depth of the microfluidic wells on the receptive material. Therefore, using the methods described herein, one can predictably and reproducibly fabricate microwells having a known depth.

In certain embodiments, the image-forming material is applied to the heat sensitive thermoplastic receptive material by one or more method comprising sputter coating, evaporation, chemical vapor deposition, pattern transfer, micro-contact printing or printing. In some embodiments, it is applied by printing. The printing can be done using any suitable printer, such as a laser or ink-jet printer or computer-controlled plotter, directly onto the thermoplastic material.

In an alternative embodiment, the image forming material is a metal. Various metals can be used as an image forming material in the methods of the disclosed invention such as gold, titanium, silver, or any other suitable metal or combination thereof. In certain embodiments, the metal is deposited by a procedure such as, for example, comprising, consisting essentially or yet further, sputter coating, evaporation or chemical vapor deposition.

In one aspect of the disclosed invention, the thermoplastic material is unstressed or pre-stressed and one which is heat sensitive and shrinks uniformly without distortion along one or two dimensions (length and width or X and Y axis). Suitable thermoplastic materials for inclusion in the methods of this invention include, for example, high molecular weight polymers such as acrylonitrile butadiene styrene (ABS), acrylic, celluloid, cellulose acetate, ethylene-vinyl acetate (EVA), ethylene vinyl alcohol (EVAL), fluoroplastics (PTFEs, including FEP, PFA, CTFE, ECTFE, ETFE), ionomers kydex, a trademarked acrylic/PVC alloy, liquid crystal polymer (LCP), polyacetal (POM or Acetal), polyacrylates (Acrylic), polyacrylonitrile (PAN or Acrylonitrile), polyamide (PA or Nylon), polyamide-imide (PAI), polyaryletherketone (PAEK or Ketone), polybutadiene (PBD), polybutylene (PB), polybutylene terephthalate (PBT), polyethylene terephthalate (PET), Polycyclohexylene Dimethylene Terephthalate (PCT), polycarbonate (PC), polyhydroxyalkanoates (PHAs), polyketone (PK), polyester polyethylene (PE), polyetheretherketone (PEEK), polyetherimide (PEI), polyethersulfone (PES), polysulfone polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), polyvinylidene chloride (PVDC) and spectralon. The materials can be unstressed or alternatively pre-stressed (“pre-shrunk”) in one dimension (uni-axially biased) such that upon application of the first material and heating, it only shrinks in the second dimension. Alternatively is can be shrunk in both directions (bi-axially biased).

In one aspect, the above methods are performed under conditions wherein the thermoplastic material is reduced in size by heating or other method known in the art such that the length and/or width of the second material is reduced by at least 20% of its original size prior to heating and increase the height of the first material is increased by at least 3 times of the area of the second material to which the first material is applied. Alternative embodiments of the methods include, but are not limited to the application of heat to reduce the size of the receptive material by at least 30%, or alternatively, at least 40%, or alternatively, at least 50%, or alternatively, at least 60%, or alternatively, at least 70%, or alternatively, at least 75%, or alternatively, at least 80%, or alternatively, at least 85%, or alternatively, at least 90%, or alternatively, at least 95%. The height of the first material alternatively can be increased by at least 3.25 times, or alternatively at least 3.5 times, or alternatively at least 3.75 times, or alternatively at least 4.0 times, or alternatively 4.25 times, or alternatively at least 4.5 times, the original height of the first material.

Diameter and size of the microwells is controlled by the loading density of the image forming material and the shrinking of the thermoplastic material upon which the image forming material is applied. The microwells of the array have substantially the same or different volumes. For the purpose of illustration only, the volume of each microwell can be from about 1×10⁶ μm³ to about 0.2 μm³, or alternatively from about 1×10⁵ mm³ to about 0.2 mm³, or alternatively from about 1×10⁴ μm³ to about 0.2 μm³, or alternatively from about 1×10³ μm³ to about 0.2 μm³, or alternatively from about 1×10² μm³ to about 0.2 μm³, or alternatively from about 10 μm³ to about 0.2 μm³, or alternatively from about 1 μm³ to about 0.2 μm³, or alternatively from about 0.5 μm³ to about 0.2 μm³, or alternatively less than about 1×10⁶ μm³, or alternatively less than about 1×10⁵ μm³, or alternatively less than about 1×10⁴ μm³, or alternatively less than about 1×10³ μm³, or alternatively less than about 1×10² mm³, or alternatively from about 10 μm³, or alternatively less than about 1 μm³.

In another aspect, each of the microwells has a diameter of from about 10 micrometers to about 2 millimeters, or alternatively from about 9 micrometers to about 2 millimeters, or alternatively from about 8 micrometers to about 2 millimeters, or alternatively from about 7 micrometers to about 2 millimeters, or alternatively from about 6 micrometers to about 2 millimeters, or alternatively from about 5 micrometers to about 2 millimeters, or alternatively from about 4 micrometers to about 2 millimeters, or alternatively less than about 9 micrometers, or alternatively less than about 8 micrometers, or alternatively less than about 7 micrometers, or alternatively less than about 6 micrometers, or alternatively less than about 5 micrometers, or alternatively less than about 4 micrometers, or alternatively less than about 3 micrometers.

In another aspect, the each of the microwells has a diameter of from about 100 micrometers to about 500 micrometers, or alternatively from about 200 micrometers to about 500 micrometers, or alternatively from about 300 micrometers to about 500 micrometers, or alternatively from about 100 micrometers to about 400 micrometers, or alternatively from about 100 micrometers to about 300 micrometers. In a yet further aspect, each of the microwells has the same or different diameter and is selected from the group consisting of about 100 micrometers, about 200 micrometers, about 300 micrometers, about 400 micrometers, and about 500 micrometers. Stated another way, the microwells will have a well diameter of about 0.0175″, or about 0.0148″, or about 0.0099″, or about 0.0049″ respectively, with the spacing of the centers of each well at about 0.0179″, or about 0.0450″, or about 0.0147″, or about 0.0117″, respectively.

With each of the above diameters for the microwells, the microwells can be the same or different depths. The microwells each may have a depth of from about 10 micrometers to about 2 millimeters, 1 or alternatively from about 9 micrometers to about 2 millimeters, or alternatively from about 8 micrometers to about 2 millimeters, or alternatively from about 7 micrometers to about 2 millimeters, or alternatively from about 6 micrometers to about 2 millimeters, or alternatively from about 5 micrometers to about 2 millimeters, or alternatively from about 4 micrometers to about 2 millimeters, or alternatively less than about 9 micrometers, or alternatively less than about 8 micrometers, or alternatively less than about 7 micrometers, or alternatively less than about 6 micrometers, or alternatively less than about 5 micrometers, or alternatively less than about 4 micrometers, or alternatively less than about 3 micrometers, or alternatively of from about 100 micrometers to about 500 micrometers, or alternatively from about 200 micrometers to about 500 micrometers, or alternatively from about 300 micrometers to about 500 micrometers, or alternatively from about 100 micrometers to about 400 micrometers, or alternatively from about 100 micrometers to about 300 micrometers. In a yet further aspect, each of the microwells has the same or different diameter and is selected from the group consisting of about 100 micrometers, about 200 micrometers, about 300 micrometers, about 400 micrometers, and about 500 micrometers.

In each of the above embodiments, the microwells can have substantially identical diameters or identical diameters, or alternatively substantially identical depths or alternatively identical depths.

The shape of the microwells can vary. The microwells can each have a contour selected from the group consisting of substantially hexangle, substantially heptangle, substantially octangle, and substantially round and the shapes can be substantially identical contours or not all of the microwells can have identical contours. In one specific embodiment, the microwells are organized in a honeycomb pattern.

To create the microwells from the reduced second thermoplastic material, the third material is prepared by a process comprising, or alternatively consisting essentially of, or yet further consists of, lithography such as soft lithography or imprint lithography from the second material. Suitable third molding materials for use in this invention include, but are not limited to one or more polymer such as polydimethylsiloxane, gelatin, agarose, polyethylene glycol, cellulose nitrate, polyacrylamide or chitosan. After creating the microwell array, the array may be washed in a series of solvents as described in more detail in the experimental section below.

After the microwells have been prepared, the wells may be coated with a material or materials that can facilitate the growth and/or differentiation of the cells or the attachment of the cells to the plate. In one aspect, the microwells are coated with a solution such as plasma to render them hydrophilic. Alternatively or in addition, the wells can be coated with materials that include, but are not limited to a growth factor selected from the group consisting of fibronectin, polylysine, gelatin or an extracellular matrix protein.

In addition, by modifying the amount of first material applied to the second material, and the heating or other method that reduces size (which in turn determines the shrinking) of the second material, one can prepare a variety of wells of different sizes and shapes, e.g., ovoid, spherical and/or square. The wells can vary in size and/or shape by multiple applications of the first material to the second and they can be the same or different from each other. For the purpose of illustration only, the first material can be applied in an amount that will produce spherical wells on the third material. The first material, in a different pattern, size or shape, can then be applied to the same second material. In this way, a plurality of wells of the same or differing size, dimension or capacity can be prepared.

This invention also provides a support or apparatus for biological, chemical or other applications (see FIGS. 3 and 4) that comprises a molding material, e.g. a polymer support such as polydimethylsiloxane, gelatin, agarose, polyethylene glycol, cellulose nitrate, polyacrylamide or chitosan, having embedded within it a plurality of microwells of the same or different size, shape or dimension such as square shape, ovoid and/or spherical.

The microwells are in relation to each other such that there is substantially no surface tension within the microwells. The size of the wells can be the same or different as described above. Examples of the plate are shown in FIGS. 3 and 4.

Microchannels can be created across the support and to the microwells and which can be connected to a source of negative pressure as shown in FIG. 3. Media can be exchanged by attaching on port or tube to a source of cell culture media and another port, to a source of negative pressure such as a vacuum pump. By applying negative pressure, the spent culture media is removed from the microwell chip and new fresh media is pulled into the microwells with minimal disruption to the cells.

This invention also provides a method for growing or culturing cells such as stem cells, by applying a cell or cell in a suitable into at least one microwell of the apparatus described above. Examples of such cells include without limitation eukaryotic and prokaryotic cells. Examples of eukaryotic cells include mammalian, e.g., murine, human or simian, adult, embryonic or iPSCs. In one aspect, the invention provides materials and methods to grow EBs. In one aspect the cells such as an embryonic stem cells, such as an animal or mammalian stem cell or cells are loaded by placing the cells in a microwell apparatus suspended in an appropriate media which is changed as necessary using methods known in the art and described above using Applicants' device. Methods for growing EBs are described in the literature such as Banerjee and Bhonde (2006) Cytotechnology 51(1): 1-5, U.S. Pat. No. 6,602,711, Dang et al. (2004) Stem Cells 22(3):275-282), Pat. Publ. No. WO 03/004626, U.S. Patent Publ. No. 2007/0148767A1, Stephen et al. (2002) Biotechnol. Bioeng. 78(4):442-453), U.S. Pat. Publ. No. 2005/0054100 and Dan et al. in “Efficiency of Embryoid Body Formation and Hematopoietic Development from Embryonic Stem Cells in Different Culture System” (2002) Biotechnol. Bioen. 78:442-253.

When it is necessary to change the media or carrier, the media is carefully removed from the microwells without disturbing the cells. Alternatively, microfluidic channels, secured by vacuum pressure, are placed on top of the cell-filled microwells (see FIG. 3). This allows perfusion of cells in a combinatorial way with chemical stimuli. This allows one to add to the microwells with the cells already loaded and easily retrieve the cells for further analysis off-chip.

### Kits

This invention further provides a kit comprising, or alternatively consisting essentially of, or yet further consisting of a) a thermoplastic material, and b) a molding material such as polydimethylsiloxane prepolymer, and c) instructions for making a plurality of wells using the thermoplastic material and the polydimethylsiloxane prepolymer. The kit may further comprise an image forming material. The kit provides instructions for making and using the apparatus described above and incorporated herein by reference.

In another aspect, the kit further comprises, or yet further consists essentially of, or yet further consists of, instructions for propagating cells in the plurality of wells. In one aspect, the instructions are for propagating eukaryotic stem cells, for example creating EBs in the plurality of wells.

The following examples are intended to illustrate, but not limit the invention.

### Example 1

The microwells can be designed in AutoCad 2002 (AutoDesk, San Rafael, Calif.). Using a Hewlett-Packard LaserJet 2200D, designs are printed onto the polystyrene thermoplastic sheets (Shrinky Dinks, K &B Innovations, North Lake, Wis.) that resemble transparencies. These thermoplastic sheets are then fed through the printer several times for additional height and/or multi-dimensional wells.

The printed sheet is placed in an oven for about 3-5 minutes at 163° Celsius. Both a standard toaster oven as well as a laboratory-grade oven can be used. Whereas slight warping can result from the toaster over, heating in the pre-heated lab oven resulted in more uniform heating. The devices were heated on a glass microscope slide for even more uniform and flat baking. It was found that the slides should not be pre-heated or they will melt the plastic.

The thermoplastic sheet naturally curls while shrinking to make the mold. Uniform heat on a flat surface will ensure that the thermoplastic sheet will re-flatten after complete shrinking. A post-bake of 7 minutes in the oven after shrinkage greatly smoothes the ink features, and helps maintain ink adhesion. Devices have been molded over ten times with the same patterning device without any noticeable deterioration in the mold.

The PDMS is poured onto the mold as in typical soft lithography, and cured at 110° Celsius for 10 minutes. The cured PDMS device is then peeled off the mold and bonded using a hand-held corona discharger (Haubert K., et al. (2006) Lab Chip Technical Note 6:1548-1549). The whole process from device design conception to working device can be completed within minutes.

### Example 2

Honeycomb microwells are an inexpensive alternative assay platform for the generation of uniform embryoid bodies derived from pluripotent stem cells, negating the use of photolithography. Honeycomb microwell patterns are created by molding polydimethylsiloxane (PDMS) onto polystyrene molds containing the honeycomb microwell patterns. PDMS microwells are then treated consecutively with non-polar and polar solvents to remove any uncross-linked PDMS monomers. Pluripotent embryonic stem cells (murine or human) are then cultured in the microwells wherein the microwells will induce the uniform aggregation of the pluripotent stem cells in each individual wells, thus allowing the controlled formation of embryoid bodies of various sizes depending on the microwell size. Due to the close spacing of the wells, analogous to that of honeycomb structures, cells which are uniformly dispersed will fall randomly into the wells and due to the curvature, depth, spacing and hydrophobic properties of PDMS of the wells will induce aggregation and eventual formation of uniform formation of embryoid bodies within each individual well; cells which land in between the wells will not be subjected to the initial uniform aggregation thus entering apoptosis, hence only cells falling within the wells will be induced to form uniform embryoid bodies. Honeycomb arrays can be scaled up to 1000 wells per chip, depending on the size of the well, and can be placed into a standard 24 well culture plate, thus yielding over 2000 embryoid bodies per 24 well culture plate. In addition, due to its' ability to integrate into standard culture plate honeycomb microwells can be adapted to most liquid handling systems for robust high throughput screening.

In one aspect, the fabrication of these microwells entail the following procedures. Desired well patterns are prepared in a drafting software such that the size and spacing of the wells accounts for the approximate 60% reduction in width and 300% increase in depth upon the heating of the prestressed polystyrene sheets which these wells are printed on, i.e. for 400, 300, 200 and 100 micron well patterns the well must have a diameter of approximately 0.0175″, 0.0148″, 0.0099″, 0.0049″ respectively with the spacing of the centers of each well at 0.0179″, 0.0450″, 0.0147″, and 0.0117″ corresponding to 400, 300, 200 and 100 micron wells. The pattern is printed and alignment of the wells onto biaxially stressed polystyrene sheets or other suitable material is performed such that with each consecutive printing of desired pattern onto the same polystyrene sheet would increase the depth and size of the honeycomb pattern wells due to the increase in ink deposition. The prestressed polystyrene sheet containing the pattern is cut out and heated to induce shrinking and pulling of the ink into microwell molds at approximate 155 degrees Celsius. The polystyrene masters are molded with polydimethylsiloxane (PDMS) or other suitable material to form PDMS honeycomb micowells.

The wells are placed closely together in autocad such that upon heating the wells pull closely together, but will not merge. Also the specific staggered arrangement of the wells are such that each well is forced into a more uniform shape by the surrounding wells. Wells are heated at from 155-165 degrees Celsius as to ensure the melting and pulling of the ink. During the heating process the chips are flipped upside down to allow further pulling of the ink due to gravity. The chips are then flipped back to prevent touching of the ink to the glass surface. Next the chip is flattened by running a spatula around the perimeter of the microwell design.

The shrinkage is approximately 60% reduction in size in the X & Y axis and approximately 160% in the Z direction when the thermoplastic material is flipped. Chips are placed in the over for approximately 10-15 minutes on a cooled glass slide. The chip is allowed to shrink into a bowl shape for about 5 minutes before they are flipped upside down this way the bowl shape will prevent the ink from touching the glass. Next after the chip is nearly flat, so that the ink is almost touching the glass, this may take about another 5 minutes, the chip is flipped over with the ink side facing up and is flattened around the perimeter with a spatula.

To induce the formation of embryoid bodies, the cell culture method should be as follows. Mold the polystyrene honeycomb microwell masters with PDMS. PDMS microwells are soaked in polar and non-polar solvents such as pentane for 12 hours, followed by a solvent change where new pentane is added and is further soaked for 12 hours, next the pentane solvent is replaced with xylene for 7 hours and is replaced with new xylene for another 12 hours, last the microwells are soaked in ethanol for 12 hours prior to use. To simplify the protocol and save time, the first solvent is generally used to swell PDMS as much as possible, then followed by de-swelling gradually. Solvents that swelled PDMS the least: water, nitromethane, dimethyl sulfoxide, ethylene glycol, perfluorotributylamine, perfluorodecalin, acetonitrile, and propylene carbonate. Solvents that swelled PDMS the most: diisopropylamine, triethylamine, pentane, and xylenes. For an example of swelling and de-swelling procedure, soak in pentane for 24 hours; pentane 7 hours; then xylene isomers plus ethylbenzene 98.5% 1-2 hours; then xylenes for 16 hours; xylenes for 7 hours; then EtOH 1-2 hours, then EtOH again for 16 hours, and finally EtOH for 7 hours. Then soak in about 1 L of sterile DI water overnight and dry at 70° C. overnight.

In order of decreasing alternatives to the solvents for this process include diisopropylamine, triethylamine, pentane, xylenes, chloroform, ether, tetrahydrofuran (THF), hexanes, trichloroethylene, n-heptane, cyclohexane, dimethoxyethane (DME), toluene, benzene, chlorobenzene, methylene chloride, t-butyl alcohol, 2-butanone, ethyl acetate, dioxane, 1-propanol, acetone, pyridine, ethyl alcohol, dimethyl carbonate, N-methylpyrrolidone (NMP), dimethylformamide (DMF), methanol, phenol, propylene carbonate, acetonitrile, perfluorotributylamine, perfluorodecalin, nitromethane, dimethylsulfoxide (DMSO), ethylene glycol, glycerol or water.

The treated PDMS honeycomb microwells are then bonded to small glass slides with the wells facing up. In one aspect, it may be desirable prior to cell culture, to dry the honeycomb microwells plasma treated to create a temporary hydrophilic surface for cell loading and is kept in 70% ethanol under UV lamp for 10 minutes. The sterile microwells are then washed with PBS/water before loading into culture plates and is covered with cell culture medium to prevent formation of bubbles. Pluripotent stem cells are then broken up into single cells either by mechanical or enzymatic digestion and is loaded onto the microwells. Cells are then uniformly dispersed within the cell culture well containing the honeycomb microwells and allowed to settle. Optimal cell concentration will be determined empirically determined based on cell line and well size.

In summary, this invention provides an assay platform for the generation of uniform embryoid body formation negating the use of photolithography. By printing microwell patterns onto prestressed polystyrene sheets and heating, honeycomb patterned microwells masters are made which will then be molded with PDMS to create PDMS honeycomb microwells. Due to the curvature, size, spacing and hydrophobicity of PDMS, the honeycomb microwells will induce embryoid body formation.

### Example 3

A modification of the culturing methodology has been devised by the inventors. hESCs die during single cell dissociation (Ungrin, D. M. et al. (2008) PLoS ONE Vol. 3, e1565). Microwells require uniform single cell suspension, hence ROCK inhibitor (ROCKi) Y27632 (Watanabe, K. et al. (2007) Nature Biotechnology, 25(6); Leverrier, Y. et al. (2001) Nature Cell Biology, Vol. 3; Shi, J. et al. (2007) Arch. Immunol. Ther. Exp., 55:61-75; and Ishizaki, T. et al. (2000) Molecular Pharmacology, 57:976-983) is used at approximately 10 μM to prevent cell death. It is within the scope of this invention to modify the amount of ROCK inhibitor from approximately 5 to 15 μM, or about 6 to 14 μM, or about 7 to 13 μM, or about 8 to 12 μM or about 9 to 11 μM, as determined by those of skill in the art. Thereafter, cells are dissociated in 10 μM ROCKi and suspended culture medium containing 10 μM ROCKi. It is within the scope of this invention to modify the amount of ROCK inhibitor from approximately 5 to 15 μM, or about 6 to 14 μM, or about 7 to 13 μM, or about 8 to 12 μM or about 9 to 11 μM, as determined by those of skill in the art.

Embryoid body formation is inhibited if hESCs adhere to the substrates, PDMS etc., thus to prevent long term cell adhesion, the honeycomb microwells are treated with BSA to block non-specific protein/cell binding (Valamehr, B. et al. (2008) PNAS 105(38):14459-14464; Kim, Y. et al. (2006) Biochemical and Biophysical Research Communications 351: 953-957; and Rothschilds, A. M., et al. (1988) Hepatology 8: 385-401). Microwells are treated with BSA at a concentration of about 5 mg/mL, or alternatively about 4 mg/mL, or alternatively about 3 mg/mL, or alternatively about 2 mg/mL, or alternatively about 1 mg/mL, or alternatively about 0.5 mg/mL and temperature of about 2° C. for 24 hours prior to cell culture application. In particular embodiment, the microwells are treated with BSA at a concentration of about 1 mg/mL and a temperature of about 2° C. for 24 hours prior to cell culture application.

### Example 4

In an extension of Examples 1, 2 and 3, there is provided an ultra-rapid fabrication and culture method utilizing a laser-jet printer to generate closely arrayed honeycomb microwells of tunable sizes for the induction of uniform EBs from single cell suspension. By printing various microwell patterns onto pre-stressed polystyrene sheets, and through heat induced shrinking, high aspect micromolds were generated. Applicants achieved rounded bottom polydimethylsiloxane (PDMS) wells not easily achievable with standard microfabrication methods, but critical to achieve spherical EBs. Furthermore, by simply controlling the size of the microwells and the concentration of the cell suspension, Applicants could control the initial size of the cell aggregate, thus influencing lineage commitment. In addition, these microwells are easily adaptable and scalable to most standard well plates and easily integrated into commercial liquid handling systems to provide an inexpensive and easy high throughput compound screening platform.

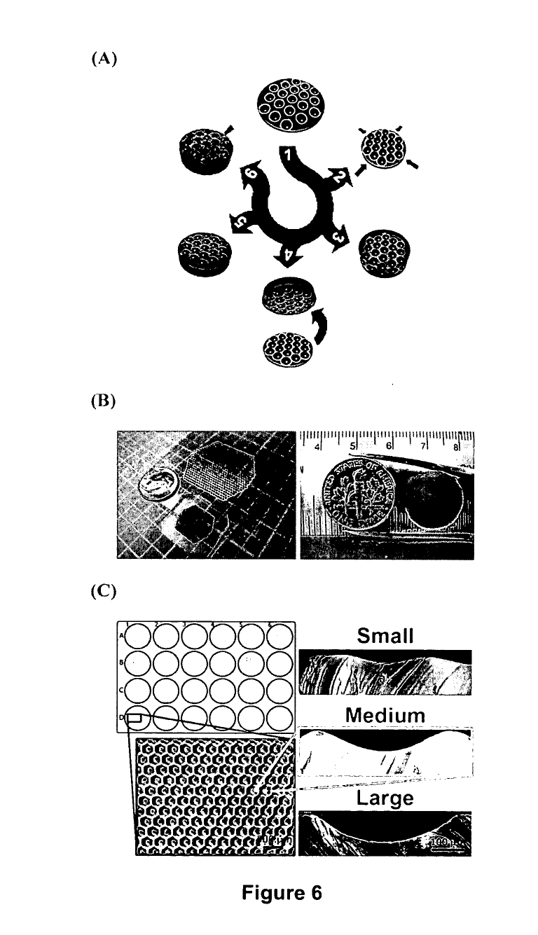

Herein, Applicants report an ultra-rapid fabrication method of closely arrayed microwells in a honeycomb configuration of customizable and well-controlled size (including diameter, depth and number of wells) negating the need for photolithography altogether. Notably, Applicants achieved rounded bottom wells not easily achievable with standard microfabrication methods but critical to achieve spherical EBs (Karp et al. (2007) Lab Chip 7:786-794). By printing various microwell patterns onto pre-stressed polystyrene (PS) sheets, and through heat induced shrinking, high aspect micromolds were generated with approximately 60% reduction in in-plane size. (Chen et al. (2008) J. Visualized Exp. DOI: 10.3791/692; Chen et al. (2008) Lab Chip 8:622-624; Grimes et al.(2008) Lab Chip 8, 170-172; Fu et al. (2009) Adv. Mater. DOI: 10.1002/adma.200902294; Long et al. (2009) Appl. Phys. Lett. 94:133501). Polydimethylsiloxane (PDMS) is then molded onto the micromolds to form honeycomb microwells (FIG. 6). The use of printable masters does not limit the fabrication of microwells to PDMS. Other polymeric substrates (e.g. polyethylene glycol (PEG) and agarose) can be likewise molded on the high aspect ratio molds via soft lithography as well (Ling et al. (2007) Lab Chip 7:756-762). To control the number of cells per well, the cells were simply loaded into the wells by pipetting various concentrations of ESCs dissociated into single-cell suspensions.

### EXPERIMENTAL

### Fabrication of Honeycomb Microwells

Honeycomb microwell patterns were drafted in the drafting software AutoCAD (AudoDesk). In order to achieve a range of microwell diameters, accounting for the 60% reduction in size after shrinking of the pre-stressed PS sheet, Applicants tested a range of varied drafting diameters: 250, 500, 750, and 890 μm. For ease of annotation, these correspond to final well sizes referenced as ‘Small’, ‘Medium’, ‘Large’ and ‘X-Large’. To minimize the spacing between wells, well patterns were placed in a staggered position as to minimize free surface area (FIG. 6A). Next, well patterns were printed onto biaxially prestressed PS sheets (Grafix Inc.) using a laser-jet printer (Hewlett Packard 2600N). These PS molds were then heated to 155° C. for approximately 5 minutes to form high-aspect micromolds (Chen et al. (2008) J. Visualized Exp. DOI: 10.3791/692; Chen et al. (2008) Lab Chip 8:622-624; Grimes et al. (2008) Lab Chip 8, 170-172; Fu et al. (2009) Adv. Mater. DOI: 10.1002/adma.200902294; Long et al. (2009) Appl. Phys. Lett. 94:133501). After molding with PDMS using standard procedures, the microwells, designed to fit in standard 24 well plates, were achieved. Notable, the shrinking process induced reflow of the toner ink to cause rounded bottoms as evidenced in the cross sections (FIG. 6C).

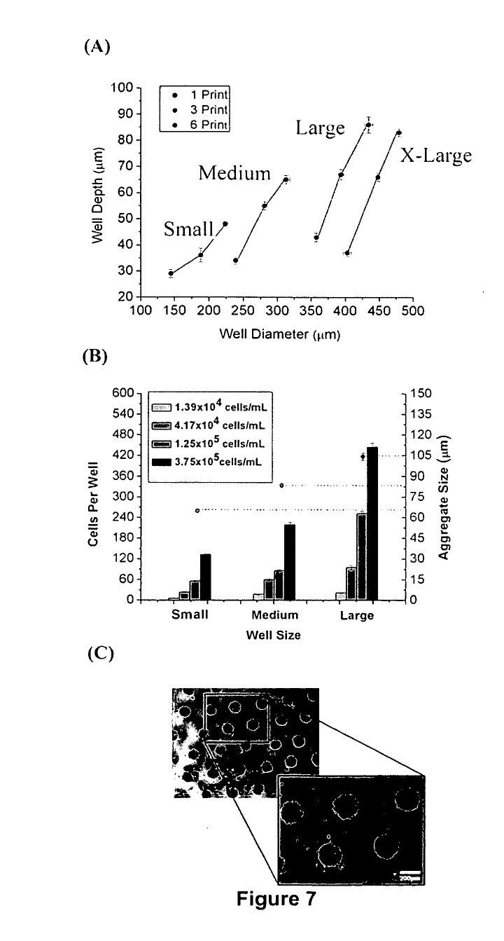

By repeated printing of the well patterns (by reinsertion into the laser-jet printer), the size and the depth of the microwell patterns could be adjusted through the increase of ink deposition (FIG. 6B). Notably, with increased number of prints, the wells grew both in diameter as well as depth (FIG. 7A). The fabrication of the honeycomb wells required the heating of the PS sheets to 155° C. Due to the difference in the shrinking temperature of the PS and that of toner melting temperature, which may vary slightly depending on the vendor, it was crucial that the devices be heated past the melting point of the toner to facilitate cohesive forces and the formation of well rounded wells. Next, to ensure the close packing of the microwells, the initial drafting patterns needed to be spaced such that, upon heating, would induce reflow of the ink without the merging of the ink droplets (FIG. 6A). The close spacing minimized the dead space between the wells and prevented the formation of non-uniform EBs on the outer perimeters due to random clusters of ES cells. Once the device was made, the toner hardened and the high-aspect micromolds could be used repeatedly to yield replicas of the same honeycomb wells.

With repeated printing, Applicants noticed an increase in well dimensions. Notably, with increased number of prints, the wells grew both in diameter as well as depth (FIG. 7A). The variation in diameter, however, was seen to have a relatively constant increase of approximately 30 μm for all three drafting diameter with each successive printing. Applicants also noticed that the depth of the microwell was limited to the surface tension of the ink toner which was evident in the diminishing increase of aspect ratio beyond six repeated prints (FIG. 7A). Thus, Applicants have seen that for fabricating microwells with depths up to 100 μm, it may not be necessary to exceed six repeated prints.

### PDMS Chemical Treatment

To prevent the potential contamination of culture medium due to uncross-linked PDMS oligomers, which may affect viability of mammalian cells, a modified method as reported by Millet et al. and Lee et al. was adopted to wash the PDMS (Millet et al. (2007) Lab Chip 7:987-994; Lee et al. (2003) Anal. Chem. 75:6544-6554). In this process, PDMS honeycomb microwells were subjected to swelling and de-swelling through a continuous stirring in 1 L of solvent, with replacement of fresh solvent at indicated intervals. Briefly, the microwells were washed consecutively with pentane for 24 h; pentane 7 h; xylenes plus ethylbenzene 98.5% 1-2 h; xylenes 16 h; xylenes 7 h; ethanol (EtOH) 1-2 h; EtOH again for 16 h, and finally EtOH for 7 h (Sigma-Aldrich). Next the PDMS was rinsed with sterile DI water and dried at 70° C. overnight.

### Cell Culture

Mouse ES cells (mESC) (ATCC) and GFP-labeled myosin heavy chain mESC, courtesy of Conklin Lab, UC San Francisco, were maintained in Knock-out Dulbecco's modification of Eagle Medium (DMEM) (Gibco) supplemented with 15% Knock-out Serum Replacement (KSR) (Gibco), 100 mg/mL penicillin-streptomycin (Invitrogen), 200 mM Glutamax (Invitrogen), 0.1 mM non-essential amino acids (NEAA) (Invitrogen), 0.1 mM β-mercaptoethanol (calbiochem) and 1000 U/mL leukemia inhibitory factor (LIF) (Chemicon) and plated on tissue cultured plates (Nunc) coated with 0.1% Gelatin (Sigma-Aldrich). To assure uniform distribution of cells during the loading process, ES cell colonies were dissociated into single cells. To this extent, cells washed twice with 1× phosphate buffered saline (PBS) (Gibco) and treated with TrypLE (Gibco) for 3 minutes. Next ES cells were gently dissociated using a P1000 pipette and spun down. ES cells were then re-suspended in EB medium, which had the same composition as ESC medium with the exclusion of LIF and KSR and supplemented with 15% fetal bovine serum (FBS).

### Microwell Loading

To load cells, the bottom of the microwells were bonded to single pieces of cover glass (Fisherbrand) using an O₂ plasma (SPI Supplies) and placed into each well of a standard 24-well plate containing 500 μL of EB medium. The initial 500 μL assisted in preventing air bubbles within the well and adhered the cover glass to the plate. Next an additional 1.0 mL of EB medium was placed into the well and was pipetted gently to remove any remaining air bubbles on the PDMS surface. ES cells were added at concentrations of 1.39×10⁴ cells/mL, 4.17×10⁴ cells/mL, 1.25×10⁵ cells/mL, and 3.75×10⁵ cells/mL (FIG. 7B). To achieve uniform EB size, it requires the uniform distribution of single cells across the microwells. Thus, using a modified suspension culture method reported by Park et al. and Ungrin et al., 1 mL of the ES cells were then gently pipetted with a P1000 and dispensed dropwise into each well of a 24-well plate (Park et al. (2007) Lab Chip 7:1018-1028; Ungrin et al. (2008) PLoS One 3, e1565). To prevent convective effects within each well of the 24-well plate which may disrupt the uniform distribution, ES cells were allowed to settle into the honeycomb microwells at room temperature for 15-30 minutes before being placed into the incubator.

To standardize the loading procedure, Applicants created a calibration curve which allowed them to calculate the optimal cell density which prevented the formation of multiple EBs per well (FIG. 7B).

To show a direct correlation between tunable well size and EB size, aggregate sizes generated from a concentration of 1.25×10⁵ cells/mL in Small, Medium and Large well were measured. Applicants found that, for a given seeding concentration, the aggregate size varied linearly correlating to the original well size. Thus, at 1.25×10⁵ cells/mL, Small, Medium and Large wells yielded aggregates approximately 65, 85 and 105 μm in diameter, ±5, 8, and 11 μm respectively (FIG. 7B).

Next to determine the initial cell number attributed to each microwell size, a custom software was used to count the number of cells distributed uniformly across each well size. Using a 1/3 dilution starting at 3.75×10⁵ cells/mL to 1.39×10⁴ cells/mL, Applicants generated a family curve for Small, Medium and Large (FIG. 7B). Notably, during the cell loading process, it was observed that the inherent non-restrictive geometry of the wells may induce the formation of multiple EBs per well at low loading densities. Due to the sparseness of the cell distribution, local colonies within a single microwell may not be able to adhere with one another, thus forming separate EBs. To solve this issue, Applicants noted two possible solutions. First, either reduce the size of the microwell while maintaining the loading density constant, thus reducing the available well space to facilitate uniform aggregation. Second, increase the loading density of the cells while maintaining the well size constant, which would ensure the uniform coverage of the wells thus allowing all the single cells to aggregate into one single EB. At the concentration Applicants used for the experiment, they observed that 90% of the wells had single EB formation.

By using the calibration curve, Applicants were also able to generate aggregates with initial cell numbers comparable to the hanging drop method, which used approximately 500 cells/well, by seeding large wells at 3.75×10⁵ cells/mL (FIG. 7B). This resulted in the formation of uniform aggregates of approximately 200 μm in diameter (FIG. 7C). Thus, by varying the microwell size Applicants were able to generate uniform aggregates of tunable sizes, with over 1,300 aggregates/cm² at the smallest size.

### EB Culture and Flow Cytometry

To observe the size dependent differentiation pattern, EB cultured in the Small, Medium, and Large microwells were transferred to a low adherent suspension culture dish (Corning) after two days (Karp et al. (2007) Lab Chip 7786-794). Oct-3/4 was used as an indicator of pluripotency (BD Biosciences) (Mitalipov et al. (2003) Biol. Reprod. 69:1785-1792). In addition GATA4, Nestin, and CD-31, were used as indicators of early germ layer development (BD Biosciences). ES cells were stained for pluripotency before plating onto microwells. Two days after culture in microwells, uniform aggregates from each corresponding well size were transferred to suspension culture and samples were taken and stained for pluripotency as well as developmental markers. Subsequently EBs derived from the three well sizes were imaged and stained on day 4 and 6 for pluripotency and differentiation.

Briefly, for Oct-3/4 staining EBs were dissociated into single cells and directly fixed with 4% formaldehyde (Gibco) and permeabilized with 0.7% Triton-X (mpbio) prior to staining. CD-31, GATA4 and Nestin were stained sequentially.

EB samples were dissociated into single cells and stained for CD-31 as an extracellular marker. Next, each sample was fixed with formaldehyde and permeabilized with Triton-X. GATA4 and Nestin were stained together as intracellular markers.

### Results and Discussion

### EB Formation and Characterization

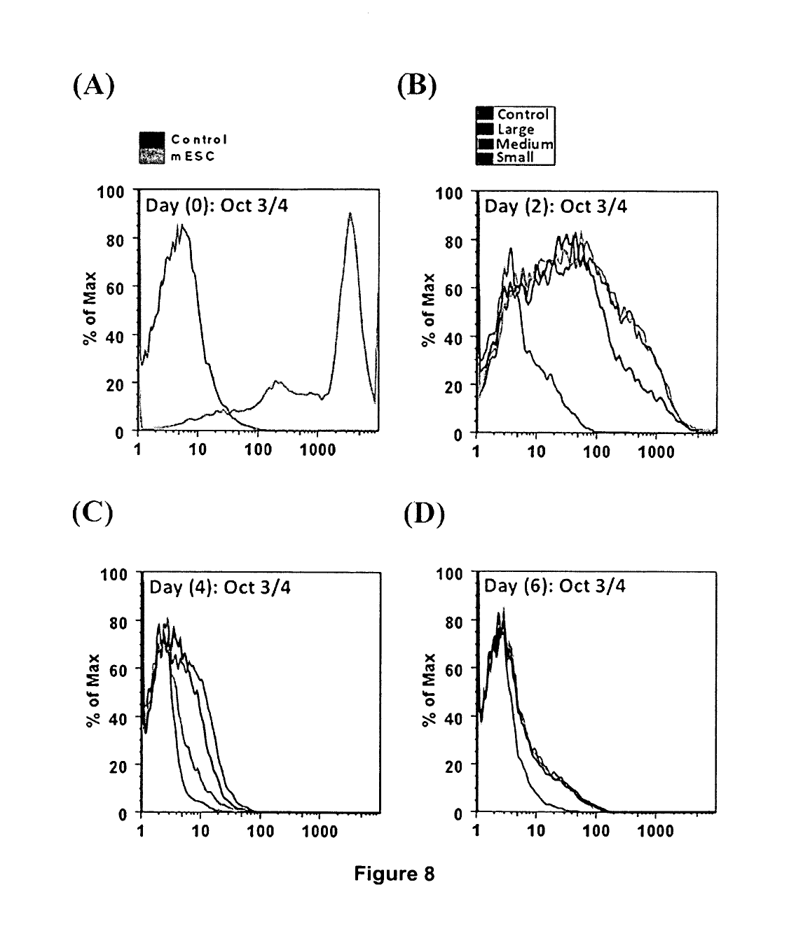

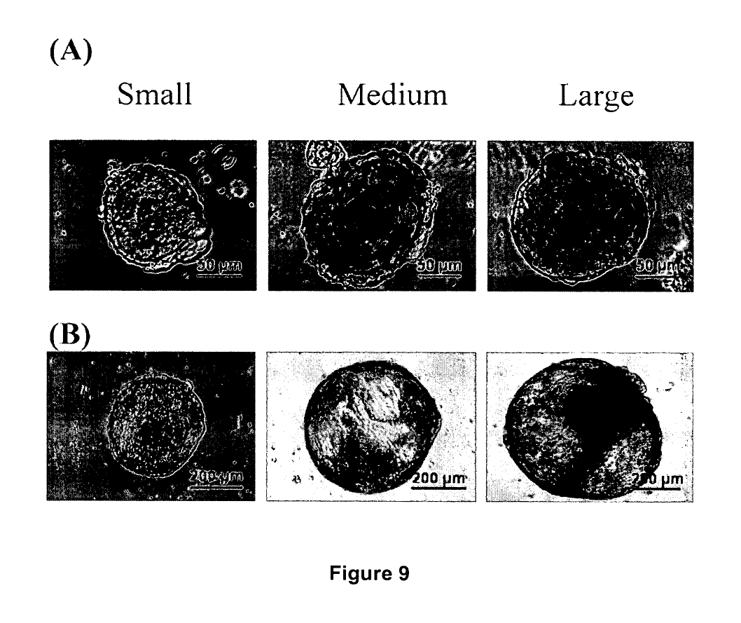

Differentiation and eventual formation of the three primary germ layers is critical for EB development. Thus, to verify that uniform aggregates generated by the honeycomb microwells can develop into viable EBs, Applicants tested for differentiation markers. To accomplish this, Small, Medium and Large microwells were seeded at the seeding density of 3.75×10⁵ cells/mL. Assessment of differentiation characteristic between on chip and post transfer was correlated with the level of Oct-3/4 expression at different stages of EB differentiation relative to the initial ES population (FIG. 8). As the aggregates developed into EBs by day 4 and 6, Applicants continued to see a gradual decay of Oct-3/4. This could be correlated with the development of the three layers and subsequent formation of cystic EBs; this could be seen by morphology by day 4 and day 6 post transfer (FIG. 9).

To quantify the expression of the primary germ layers, flow cytometry (FACS ARIA) analysis was performed. For the formation of the endoderm and ectoderm layer, Applicants chose GATA4 and Nestin, respectively (Wiese et al. (2004) Cell. Mol. Life Sci. 61:2510-2522; Arceci et al. (1993) Mol. Cell. Biol. 13, 2235-2246). These markers have been shown to be indicative of endoderm and ectoderm development and have been correlated with EB size (Park et al. (2007) Lab Chip 7:1018-1028). Applicants also stained for CD-31 (also known as PCAM1). While CD-31 is an indicator of mesoderm development and early formation of endothelial progenitors, it has also shown to be expressed in ES cell populations and downregulated during the first three days of differentiation and subsequently upregulated by day 4 of EB formation (Vittet et al. (1996) Blood 88:3424-3431; DeLisser et al. (1994) Immunol. Today 15:490-495).

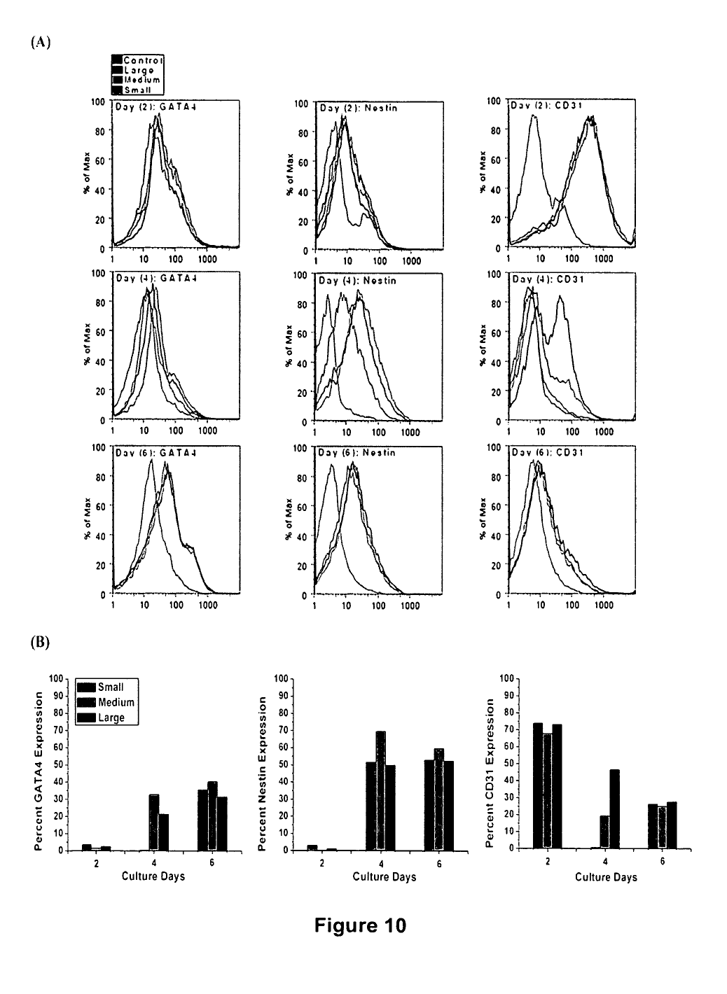

As predicted, day 2 analysis indicated a lack of both GATA4 and Nestin in small, intermediate and large aggregate populations (FIGS. 10A and 10B). CD-31, however, which is expressed in pluripotent ES cells and gradually downregulated during the first three days of EB formation, was present in all three aggregate populations at day 2, indicating the initial stages of EB formation (FIG. 10C). By day 4, both intermediate and large EB populations showed an upregulation of GATA 4 and Nestin (FIG. 10). The EB population derived from the Small wells correlating to initial aggregates of approximately 130 cells (FIG. 7B), however, showed a lack of both endoderm and mesoderm as indicated by the absence of GATA4 and CD-31. Previously, Park et al., had reported that monolayers derived from EBs smaller than 100 μm and were then subjected to lineage specific differentiation, showed a preferentially higher ectoderm expression. Here, Applicants report that this observation is seen as early as day 4 of EB development as indicated by high Nestin expression relative to other markers (FIG. 10). Applicants also observed that the cells within the largest EB aggregates showed a preferentially higher retained expression of CD-31 relative to the small and intermediate EB populations by day 4.

By day 6, Applicants noticed that all three EB populations had expressed uniform levels of GATA4, Nestin, and CD-31 (FIG. 10). Applicants also observed that by day 6, all EB populations seemed to appear to have developed the characteristic cystic center based on morphology, however, also retained a slight size difference (FIG. 9B). This led Applicants to believe that the preferential bias in lineage specification previously reported occurs during the EB formation (Park et al. (2007) Lab Chip 7:1018-1028). Thus, based on Applicants' analysis and data previously reported, Applicants believe that the tunable geometry in addition to the simple fabrication method, honeycomb microwells can be used as a possible method for the enrichment of lineage-specific tissue derivation. Further, by exploiting the preferential differences in lineage commitment, seen as early as day 4, EBs derived from microwells may be selected for further directed differentiation.

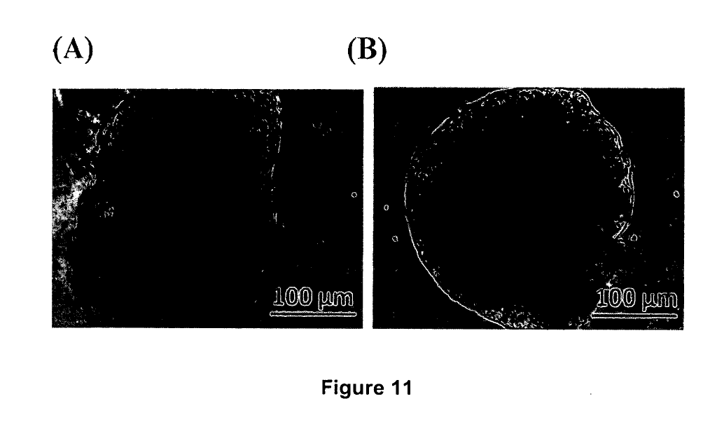

Lastly, to show that EBs derived from microwells are healthy and can develop into functional tissue level organization, Applicants compared the ability of EBs derived from microwells and those from the traditional hanging drop method to form beating cardiomyocytes. To accomplish this, large wells seeded at 3.75×10⁵ cells/mL were induced to form beating cardiomyocytes by transferring the EB colonies from suspension culture to gelatin coated culture plates after two days. Using a GFP labeled myosin heavy chain reporter gene as an indicator of cardiac tissue, fluorescence imaging was taken of the beating colonies (FIG. 11). Applicants observed that EBs derived from microwells were able to develop into beating cardiomyocytes by day 14 (FIG. 11A). In addition, the GFP expression from populations derived from microwells (FIG. 11A) were similar to those derived from the traditional hanging drop method (FIG. 11B) (data not shown). Thus, Applicants conclude that EBs derived from honeycomb microwell exhibit normal differentiation patterns. Furthermore, they are able to form functional tissue level organization comparable to traditional method.

### CONCLUSIONS

Applicants report a novel method for the fabrication of honeycomb microwells utilizing a laser-jet printer and replica molding. In addition, Applicants have shown functional application of the devices for the induction of uniform EBs of tunable sizes. By printing microwell patterns onto biaxially-stressed PS sheets and through heat induced shrinking, high aspect micromolds were generated. By molding PDMS onto the PS masters, honeycomb-shaped microwells were formed. In addition, through the inherent fabrication method, Applicants were able to generate rounded bottom wells which facilitated the formation of spherical EBs and which were potentially much less restrictive to diffusive transport. By varying the size of the honeycomb wells, Applicants were able to control the initial number of cell aggregates thus enabling control of the rate of EB growth and differentiation, which has been shown to affect lineage commitment. Notably, honeycomb microwells could be integrated into standard cell culture plates providing a low-cost, robust method of high-throughput EB culture applicable in both academic and industrial settings. Additionally, with the application of Rho-associated kinase (ROCK) inhibitors, which permitted the single cell dissociation of hESCs, this approach is also extensive to hESCs and induced pluripotent stem (iPS) cells (Watanabe et al. (2007) Nat. Biotechnol. 25:681-686; Ishizaki et al. (2000) Mol. Pharmacol. 57:976-983; Claassen et al. (2009) Mol. Reprod. Dev. 76:722-732). As such, this technology is a useful tool for a large range of applications.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A microwell array (1) comprising a plurality of microwells (2, 6, 7, 8) on a hydrophobic surface wherein the microwells each is substantially proximate to each of its adjacent microwells, and the average volume of the microwells is from about 1000 μm3 to about 10 mm3.

2. The microwell array (1) of claim 1, wherein the hydrophobic surface lacks surface tension.

3. The microwell array (1) of claim 1, wherein the microwells each has a distance to an adjacent microwell, wherein the distance is substantially the same as or slightly greater than the sum of the radiuses of both of the microwells.

4. The microwell array (1) of claim 1, wherein the microwells each has a volume of from about 1×106 μm3 to about 0.2 mm3.

5. The microwell array (1) of claim 1, wherein the microwells each has a diameter of from about 10 micrometers to about 2 millimeters.

6. The microwell array (1) of claim 1, wherein the microwells each has a diameter of from about 100 micrometers to about 500 micrometers.

7. The microwell array (1) of claim 1, wherein the microwells each has a diameter selected from the group consisting of about 100 micrometers, about 200 micrometers, about 300 micrometers, about 400 micrometers, and about 500 micrometers.

8. The microwell array (1) of claim 1, wherein the microwells each has a depth of from about 10 micrometers to about 2 millimeter.

9. The microwell array (1) of claim 8, wherein the microwells each has a depth of from about 100 micrometers to about 500 micrometers.

10. The microwell array (1) of claim 1, wherein the microwells have substantially identical diameters.

11. The microwell array (1) of claim 1, wherein not all of the microwells have identical diameters.

12. The microwell array (1) of claim 1, wherein the microwells have substantially identical depths.

13. The microwell array (1) of claim 1, wherein not all of the microwells have identical depths.

14. The microwell array (1) of claim 1, wherein the microwells each has a contour selected from the group consisting of substantially hexangle, substantially heptangle, substantially octangle, and substantially round.

15. The microwell array (1) of claim 1, wherein the microwells have substantially identical contours.

16. The microwell array (1) of claim 1, wherein not all of the microwells have identical contours.

17. The microwell array (1) of claim 1, wherein the microwells are organized in a honeycomb pattern.

18. The microwell array (1) of claim 1, wherein the array is made of a receptive material or a material comprising one or more of polydimethylsiloxane, gelatin, agarose, polyethylene glycol, cellulose nitrate, polyacrylamide or chitosan.

19. The microwell array (1) of claim 18, wherein the receptive material is a heat-shrunk thermoplastic material.

20. The microwell array (1) of claim 19, wherein the receptive material is selected from the group consisting of acrylonitrile butadiene styrene, acrylic, celluloid, cellulose acetate, ethylene-vinyl acetate, ethylene vinyl alcohol, fluoroplastics, ionomers kydex, a trademarked acrylic/PVC alloy, liquid crystal polymer, polyacetal, polyacrylates, polyacrylonitrile, polyamide, polyamide-imide, polyaryletherketone, polybutadiene, polybutylene, polybutylene terephthalate, polyethylene terephthalate, Polycyclohexylene Dimethylene Terephthalate, polycarbonate, polyhydroxyalkanoates, polyketone, polyester polyethylene, polyetheretherketone, polyetherimide, polyethersulfone, polysulfone polyethylenechlorinates, polyimide, polylactic acid, polymethylpentene, polyphenylene oxide, polyphenylene sulfide, polyphthalamide, polypropylene, polystyrene, polysulfone, polyvinyl chloride, polyvinylidene chloride, and spectralon.

21. The microwell array of claim 19, wherein the receptive material is polystyrene.

22. The microwell array of claim 19, wherein the material is polydimethylsiloxane.

23. A microwell plate (20) comprising one or more of the microwell arrays of claim 1.

24. A method for preparing a microwell array comprising a plurality of microwells comprising:
a) applying an image-forming material to a surface of an unstressed or a pre-stressed thermoplastic material in a designed pattern comprising a plurality of filled areas, wherein the filled areas each is substantially proximate to each of its adjacent filled areas and the average area of the filled areas is from about 1000 μm2 to about 20 mm2;
b) reducing the area of the surface of the thermoplastic material by at least about 60%; and
c) preparing the microwell array via lithography on a material having a hydrophobic surface.

25. The method of claim 24, wherein the image-forming material is a liquid containing one or more selected from the group consisting of pigment, dye, and combinations thereof.

26. The method of claim 24, wherein the image-forming material is one or more selected from the group consisting of an ink, a protein, a metal, a colloid, a dielectric material, a paste, and combinations thereof.

27. The method of claim 24, further comprising step d) treating the thermoplastic material consecutively with non-polar and polar solvents to remove any uncross-linked monomers.

28. The method of claim 24, wherein the image-forming material is applied to the unstressed or pre-stressed material by one or more methods selected from the group consisting of sputter coating, evaporation, chemical vapor deposition, pattern transfer, micro-contact printing and printing.

29. The method of claim 24, further comprising repeating step a) two or more times prior to performing step b).

30. The method of claim 24, wherein the filled areas each has a shape selected from the group consisting of substantially hexangle, substantially heptangle, substantially octangle, and substantially round.

31. The method of claim 24, wherein the filled areas each has a diameter of from about 100 micrometers to about 500 micrometers after step b).

32. The method of claim 24, wherein the filled areas each has a depth of from about 10 micrometers to about 1 millimeter after step b).

33. The method of claim 24, wherein the filled areas each has a depth of from about 100 micrometers to about 500 micrometers after step b).

34. The method of claim 24, wherein the filled areas are organized in a honeycomb pattern.

35. The method of claim 24, wherein the reducing of step b) is by at least about 80%.

36. The method of claim 24, wherein the pre-stressed thermoplastic material is uni-axially biased prior to performing step a).

37. The method of claim 24, wherein the pre-stressed thermoplastic material is bi-axially biased prior to performing step a).

38. The method of claim 24, wherein the unstressed or pre-stressed thermoplastic material is a heat sensitive thermoplastic material.

39. The method of claim 24, wherein the reducing of step b) is by heating.

40. The method of claim 24, wherein the lithography of step c) comprises soft lithography or imprint lithography.

41. The method of claim 24, wherein the material of step c) comprises polydimethylsiloxane.

42. A microwell plate (20) comprising one or more microwell arrays of claim 1.

43. The microwell plate (20) of claim 42, wherein the plate comprises at least one input channel (22) and at least one output channel (24), and a channel (26) connecting the input and output channel such that liquid can be exchanged from a microwell array.

44. A method for preparing a microwell array comprising a plurality of microwells comprising:
a) etching a designed pattern into a hydrophobic surface of an unstressed or a pre-stressed material, which designed pattern comprises a plurality of filled areas, wherein the filled areas each is substantially proximate to each of its adjacent filled area and the average area of the filled areas is from about 1000 μm2 to about 20 mm2; and
b) reducing the area of the surface of the pre-stressed material by at least about 60%, thereby preparing the microwell array.

45. A microwell array prepared by the method of claim 44.

46. A microwell plate (20) comprising one or more of the microwell arrays prepared by the method of claim 44.

47. The microwell plate (20) of claim 44, wherein the plate comprises at least one input channel (22) and at least one output channel (24), and a channel (26) connecting the input and output channel such that liquid is exchanged between the microwells.

48. A method for culturing a cell comprising growing the cell in a microwell of the microwell array of claim 1.

49. The method of claim 47, wherein the cell is an isolated prokaryotic or eukaryotic cell.

50. The method of claim 49, wherein the cell is an isolated eukaryotic cell.

51. The method of claim 50, wherein the isolated eukaryotic cell is an isolated stem cell.

52. The method of claim 51, wherein the isolated stem cell is selected from the group consisting of an embryonic stem cell, a pluriopotent stem cell, a somatic stem cell, an iPS stem cell and combinations thereof.

53. The method of claim 51 or 52, wherein the isolated stem cell is an animal stem cell.

54. The method of claim 53, wherein the animal stem cell is of the group of mammalian, simian, bovine or murine.

55. The method of claim 53, wherein the animal stem cell is a human stem cell.

56. A method for preparing an embryoid body, comprising the steps of:
placing a solution comprising an isolated embryonic stem cell on a microwell of the microwell array of claim 1; and
allowing the cell to settle in the microwell and grow into an embryoid body.

57. The method of claim 56, further comprising removing the embryoid body from the microwell.

58. A method for assaying a potential agent for the ability to affect growth and/or differentiation of an isolated stem cell, comprising the steps of:
placing a solution comprising an isolated stem cell and an agent in a microwell of the microwell plate of claim 23,
allowing the cell to settle on the plate and grow and/or differentiate; and
assaying for the agent's ability to affect growth and/or differentiation of the cell.

59. A kit for culturing a cell, comprising a microwell plate (20) having one or more microwell arrays (1), which microwell arrays each comprises a plurality of microwells (2, 6, 7, 8) on a hydrophobic surface wherein the microwells each is substantially proximate to each of its adjacent microwells, and the average volume of the microwells is from about 1000 μm3 to about 10 mm3, and instructions for using the microwell plate to culturing a cell.
